(19) United States
(12) Patent Application Publication
(10) Pub. No.: US 2011/0077212 A1
(43) Pub. Date: Mar. 31, 2011

(54) THERAPEUTIC USES OF SGLT2 INHIBITORS

(75) Inventors: Brian Seed, Boston, MA (US); Jordan Mechanic, Sunnyvale, CA (US)

(73) Assignee: Theracos, Inc., Sunnyvale, CA (US)

(21) Appl. No.: 12/888,326

(22) Filed: Sep. 22, 2010

Related U.S. Application Data
(60) Provisional application No. 61/245,941, filed on Sep. 25, 2009.

(51) Int. Cl. A61K 31/70; A61P 7/10; A61P 3/10

(52) U.S. Cl. 514/23

(57) ABSTRACT

Provided are methods of using one or more SGLT2 inhibitors, independently or in combination, for treating edema or reducing fluid retention. The invention also provides methods of using one or more SGLT2 inhibitors for the preparation of a medicament for treating edema or fluid retention. Methods are also provided for treating diabetes with an amount of one or more SGLT2 inhibitors and one or more PPAR-gamma agonists.

### CROSS-REFERENCE TO RELATED APPLICATIONS

This application claims the benefit of U.S. Provisional Application Ser. No. 61/245,941 filed Sep. 25, 2009, the disclosure of which is incorporated herein by reference.

### STATEMENT AS TO RIGHTS TO INVENTIONS MADE UNDER FEDERALLY SPONSORED RESEARCH AND DEVELOPMENT

NOT APPLICABLE

### BACKGROUND OF THE INVENTION

The sodium-dependent glucose cotransporter 2 (SGLT2) is localized in the renal proximal tubule and is reportedly responsible for the majority of glucose reuptake by the kidneys. Studies suggest that inhibition of SGLT2 may be a useful approach to treating hyperglycemia by increasing the amount of glucose excreted in the urine (Arakawa K, et al., Br J Pharmacol 132:578-86, 2001; Oku A, et al., Diabetes 48:1794-1800, 1999). The potential of this therapeutic approach is further supported by findings that mutations in the SGLT2 gene occur in cases of familial renal glucosuria, an apparently benign condition characterized by urinary glucose excretion in the presence of normal serum glucose levels and the absence of general renal dysfunction, electrolyte imbalance or other disease (Santer R, et al., J Am Soc Nephrol 14:2873-82, 2003; Magen D, et al., Kidney Int 67:34-41, 2005; Calado J, et al., Kidney Int 69:852-5, 2006). Therefore, compounds which inhibit SGLT2 are currently under investigation for use as antidiabetic drugs (Isaji M, Curr Opin Investig Drugs 8:285-92, 2007; Jabbour S A and Goldstein B J, Int J Clin Pract 62:1279-84, 2008; Washburn W N, J Med Chem 52:1785-94, 2009).

Surprisingly, we have discovered that SGLT2 inhibitors are also useful for reducing fluid retention, particularly edema induced by agonists of peroxisome proliferator-activated receptor gamma (PPAR-gamma). Members of the nuclear receptor supergene family, the PPARs are ligand-activated transcription factors that participate in the regulation of metabolism, development and cellular differentiation. There are three main PPAR forms transcribed from different genes: PPAR-alpha, PPAR-delta/beta and PPAR-gamma. Agonists of PPAR-gamma have been widely studied for their therapeutic potential, and there are currently two marketed PPAR-gamma agonists of the thiazolidinedione class that are in use for the treatment of type 2 diabetes: rosiglitazone and pioglitazone. Still other PPAR-active agents are described in the literature as PPAR modulators based on their mixed pattern of activation of the transcription factor. PPAR-gamma agonists, or modulators, can cause increased plasma volume and fluid retention (Wang F, et al., Diabetes Technol Ther 4:505-14, 2002; Mudaliar S, et al., Endocr Pract 9:406-16, 2003). While some rodent studies suggest that fluid retention induced by PPAR-gamma agonists may involve the epithelial sodium channel ENaC, somewhat conflicting results were obtained in mice versus rats (Guan Y, et al., Nature Med 11:861-6, 2005; Chen L, et al., J Pharmacol Exp Ther 312:718-25, 2005), and therefore the mechanisms remain poorly elucidated. Because fluid retention is a potentially serious side effect, the use of PPAR-gamma agonists to treat type 2 diabetes is restricted in certain patient populations. For example, the labels of rosiglitazone and pioglitazone include black box warnings indicating increased risk for congestive heart failure. Our finding that SGLT2 inhibitors can reduce PPAR-gamma agonist-induced fluid retention may lead to the use of SGLT2 inhibitors in combination with PPAR-gamma agonists in patients at risk for development of edema.

### BRIEF SUMMARY OF THE INVENTION

The present invention provides methods of using one or more SGLT2 inhibitors, independently or in combination for reducing fluid retention in a subject. The invention also provides methods of using one or more SGLT2 inhibitors for the preparation of a medicament for treating fluid retention or edema, typically associated with the use of a PPAR-gamma agonist.

The present invention provides methods of treating type 2 diabetes, comprising administering to a subject in need thereof, a therapeutically effective amount of a combination of one or more SGLT2 inhibitors and one or more PPAR-gamma agonists, wherein said subject is at risk for development of a disease or condition associated with fluid retention.

In still another aspect, the present invention provides a method for the treatment of type 2 diabetes, comprising administering to a subject in need thereof, a therapeutically effective amount of a combination of an SGLT2 inhibitor and a PPAR-gamma agonist, wherein said subject has a disease or condition associated with fluid retention.

In another aspect, the invention provides pharmaceutical compositions comprising a mixture of therapeutically effective amounts of one or more SGLT2 inhibitors and one or PPAR-gamma agonists.

### BRIEF DESCRIPTION OF THE DRAWINGS

FIG. 1 illustrates the effects of an SGLT2 inhibitor on plasma volume in SD rats receiving pioglitazone.

FIG. 2 illustrates the change in plasma volume upon treatment with an SGLT2 inhibitor in SD rats receiving pioglitazone.

FIG. 3 illustrates the effects of an SGLT2 inhibitor on body weights of SD rats receiving pioglitazone.

FIG. 4 illustrates the change in body weights upon treatment with an SGLT2 inhibitor in SD rats receiving pioglitazone.

### DETAILED DESCRIPTION OF THE INVENTION

### Definitions

As used herein, unless otherwise indicated, the term “alkyl” alone or in combination refers to a monovalent saturated aliphatic hydrocarbon radical having the indicated number of carbon atoms. The radical may be a linear or branched chain. Illustrative examples of alkyl groups include, but are not limited to, methyl, ethyl, n-propyl, n-butyl, n-pentyl, n-hexyl, isopropyl, isobutyl, isopentyl, amyl, sec-butyl, tert-butyl, tert-pentyl, n-heptyl, n-octyl, n-nonyl, n-decyl, n-dodecyl, n-tetradecyl, n-hexadecyl, n-octadecyl, n-eicosyl and the like. Preferred alkyl groups include methyl, ethyl, n-propyl and isopropyl.

As used herein, unless otherwise indicated, the term “alkenyl” alone or in combination refers to a monovalent aliphatic hydrocarbon radical having the indicated number of carbon atoms and at least one carbon-carbon double bond. The radical may be a linear or branched chain, in the E or Z form. Illustrative examples of alkenyl groups include, but are not limited to, vinyl, 1-propenyl, 2-propenyl, isopropenyl, 1-butenyl, 2-butenyl, isobutenyl, 2-methyl-1-propenyl, 1-pentenyl, 2-pentenyl, 4-methyl-2-pentenyl, 1,3-pentadienyl, 2,4-pentadienyl, 1,3-butadienyl and the like. Preferred alkenyl groups include vinyl, 1-propenyl and 2-propenyl.

As used herein, unless otherwise indicated, the term “alkynyl” alone or in combination refers to a monovalent aliphatic hydrocarbon radical having the indicated number of carbon atoms and at least one carbon-carbon triple bond. The radical may be a linear or branched chain. Illustrative examples of alkynyl groups include, but are not limited to, ethynyl, 1-propynyl, 2-propynyl, 1-butynyl, 2-butynyl, 1-pentynyl, 2-pentynyl, 3-methyl-1-pentynyl, 3-pentynyl, 1-hexynyl, 2-hexynyl, 3-hexynyl and the like. Preferred alkynyl groups include ethynyl, 1-propynyl and 2-propynyl.

As used herein, unless otherwise indicated, the term “cycloalkyl” alone or in combination refers to a monovalent alicyclic saturated hydrocarbon radical having three or more carbons forming a carbocyclic ring. Illustrative examples of cycloalkyl groups include, but are not limited to, cyclopropyl, cyclobutyl, cyclopentyl, cyclohexyl, cycloheptyl, cyclooctyl, cyclononyl and the like.

As used herein, unless otherwise indicated, the term “cycloalkenyl” alone or in combination refers to a monovalent alicyclic hydrocarbon radical having three or more carbons forming a carbocyclic ring and at least one carbon-carbon double bond. Illustrative examples of cycloalkenyl groups include, but are not limited to, cyclopentenyl, cyclohexenyl and the like.

As used herein, unless otherwise indicated, the term “alkylene refers to a divalent hydrocarbon radical that is formed by removal of a hydrogen atom from an alkyl radical, as such term is defined above.

As used herein, unless otherwise indicated, the term “aryl” alone or in combination refers to a monovalent aromatic hydrocarbon radical having six to ten carbon atoms forming a carbocyclic ring. Illustrative examples of aryl groups include, but are not limited to, phenyl, naphthyl, tetrahydronaphthyl, indanyl and the like. Preferred aryl groups are phenyl and naphthyl, optionally mono- or disubstituted by identical or different substituents selected from halo, cyano, C₁-C₃ alkyl, C₃-C₆ cycloalkyl, difluoromethyl, trifluoromethyl, C₁-C₃ alkoxy, difluoromethoxy and trifluoromethoxy.

As used herein, the term “halo” means a monovalent halogen radical or atom selected from fluoro, chloro, bromo and iodo. Preferred halo groups are fluoro, chloro and bromo.

As used herein, unless otherwise indicated, the term “heterocycloalkyl” alone or in combination refers to a cycloalkyl group as defined above in which one or more carbons in the ring is replaced by a heteroatom selected from N, S and O. Accordingly, a C₃-C₆ heterocycloalkyl group is a three- to six-membered ring in which one or more of the carbon atom ring vertices has been replaced by N, S or O. Illustrative examples of heterocycloalkyl groups include, but are not limited to, pyrrolidinyl, tetrahydrofuranyl, piperazinyl, tetrahydropyranyl, and the like.

As used herein, unless otherwise indicated, the term “heteroaryl” alone or in combination refers to a monovalent aromatic heterocyclic radical having two to nine carbons and one to four heteroatoms selected from N, S and O forming a five- to ten-membered monocyclic or fused bicyclic ring. Illustrative examples of heteroaryl groups include, but are not limited to, pyridyl, pyridazinyl, pyrazinyl, pyrimidinyl, triazinyl, quinolinyl, isoquinolinyl, quinoxalinyl, quinazolinyl, benzotriazinyl, benzimidazolyl, benzopyrazolyl, benzotriazolyl, benzisoxazolyl, isobenzofuryl, isoindolyl, indolizinyl, thienopyridinyl, thienopyrimidinyl, pyrazolopyrimidinyl, imidazopyridines, benzothiaxolyl, benzofuranyl, benzothienyl, indolyl, isothiazolyl, pyrazolyl, indazolyl, imidazolyl, triazolyl, tetrazolyl, oxazolyl, isoxazolyl, oxadiazolyl, thiadiazolyl, pyrrolyl, thiazolyl, furyl, thienyl and the like. Five- or six-membered monocyclic heteroaryl rings include: tetrahydrothiophenyl, pyridyl, pyridazinyl, pyrazinyl, pyrimidinyl, triazinyl, isothiazolyl, pyrazolyl, imidazolyl, triazolyl, tetrazolyl, oxazolyl, isoxazolyl, oxadiazolyl, thiadiazolyl, pyrrolyl, thiazolyl, furyl, thienyl and the like. Eight- to ten-membered bicyclic heteroaryl rings having one to four heteroatoms include: quinolinyl, isoquinolinyl, quinoxalinyl, quinazolinyl, benzotriazinyl, benzimidazolyl, benzopyrazolyl, benzotriazolyl, benzisoxazolyl, isobenzofuryl, isoindolyl, indolizinyl, thienopyridinyl, thienopyrimidinyl, pyrazolopyrimidinyl, imidazopyridinyl, benzothiaxolyl, benzofuranyl, benzothienyl, indolyl, indazolyl, and the like.

As used herein, unless otherwise indicated, the term “alkoxy” alone or in combination refer to an aliphatic radical of the form alkyl-O—, wherein alkyl is as defined above. Illustrative examples of alkoxy groups include, but are not limited to, methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy, octoxy and the like. Preferred alkoxy groups include methoxy and ethoxy.

As used herein, unless otherwise indicated, the term “cycloalkoxy” alone or in combination refer to an aliphatic radical of the form cycloalkyl-O—, wherein cycloalkyl is as defined above. Illustrative examples of cycloalkoxy groups include, but are not limited to, cyclopropoxy, cyclobutoxy and cyclopentoxy.

As used herein, unless otherwise indicated, the term “heterocycloalkoxy” alone or in combination refer to an aliphatic radical of the form heterocycloalkyl-O—, wherein heterocycloalkyl is as defined above. Illustrative examples of heterocycloalkoxy groups include, but are not limited to, tetrahydrofuranoxy, pyrrolidinoxy and tetrahydrothiophenoxy.

As used herein, unless otherwise indicated, the term “haloalkyl” refers to an alkyl radical as described above substituted with one or more halogens. Illustrative examples of haloalkyl groups include, but are not limited to, chloromethyl, dichloromethyl, fluoromethyl, difluoromethyl, trifluoromethyl, 2,2,2-trichloroethyl and the like.

As used herein, when a particular position in a compound is designated as being “deuterated” (the element deuterium is represented by the letter “D” in chemical structures and formulas and indicated with a lower case “d” in chemical names, according to the Boughton system), it is understood that the abundance of deuterium at that position is substantially greater than the natural abundance of deuterium, which is 0.015%. In certain embodiments, a composition has a minimum isotopic enrichment factor of at least 5 (0.075% deuterium incorporation), e.g., at least 10 (0.15% deuterium incorporation). In other embodiments, a composition has an isotopic enrichment factor of at least 50 (0.75% deuterium incorporation), at least 500 (7.5% deuterium incorporation), at least 2000 (30% deuterium incorporation), at least 3000 (45% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), or at least 6600 (99% deuterium incorporation).

As used herein, the terms “treating” and “treatment” refer to delaying the onset of, retarding or reversing the progress of, or alleviating or preventing either the disease or condition to which the term applies, or one or more symptoms of such disease or condition.

As used herein, the term “administering” means oral administration, administration as a suppository, topical contact, intravenous, intraperitoneal, intramuscular, intralesional, intranasal or subcutaneous administration, or the implantation of a slow-release device, e.g., a mini-osmotic pump, to a subject. Administration is by any route including parenteral, and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, and the like.

As used herein, the term “prodrug” refers to a precursor compound that, following administration, releases the biologically active compound in vivo via some chemical or physiological process (e.g., a prodrug on reaching physiological pH or through enzyme action is converted to the biologically active compound). A prodrug itself may either lack or possess the desired biological activity.

As used herein, the term “compound” refers to a molecule produced by any means including, without limitation, synthesis in vitro or generation in situ or in vivo.

As used herein, the phrase “consisting essentially of” refers to the genera or species of active pharmaceutical agents included in a method or composition, as well as any excipients inactive for the intended purpose of the methods or compositions. In some embodiments, the phrase “consisting essentially of” expressly excludes the inclusion of one or more additional active agents other than an SGLT2 inhibitor and a PPAR-gamma agonist.

The terms “controlled release,” “sustained release,” “extended release,” and “timed release” are intended to refer interchangeably to any drug-containing formulation in which release of the drug is not immediate, i.e., with a “controlled release” formulation, oral administration does not result in immediate release of the drug into an absorption pool. The terms are used interchangeably with “nonimmediate release” as defined in Remington: The Science and Practice of Pharmacy, 21^{st} Ed., Gennaro, Ed., Lippencott Williams & Wilkins (2003). As discussed therein, immediate and nonimmediate release can be defined kinetically by reference to the following equation:

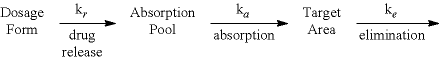

The “absorption pool” represents a solution of the drug administered at a particular absorption site, and kᵣ, kₐ and kₑ are first-order rate constants for (1) release of the drug from the formulation, (2) absorption, and (3) elimination, respectively. For immediate release dosage forms, the rate constant for drug release kᵣ is far greater than the absorption rate constant kₐ. For controlled release formulations, the opposite is true, i.e., kᵣ<<kₐ, such that the rate of release of drug from the dosage form is the rate-limiting step in the delivery of the drug to the target area.

The terms “sustained release” and “extended release” are used in their conventional sense to refer to a drug formulation that provides for gradual release of a drug over an extended period of time, for example, 12 hours or more, and that preferably, although not necessarily, results in substantially constant blood levels of a drug over an extended time period.

As used herein, the term “delayed release” refers to a pharmaceutical preparation that passes through the stomach intact and dissolves in the small intestine.

The terms “congestive heart failure” and “CHF” as used herein refer to heart failure caused by impaired pumping capability of the heart that is not keeping up with the metabolic needs of body tissues and organs; it is associated with abnormal retention of water and sodium. Decreased cardiac output causes an increase in the blood volume within the vascular system. Congestion within the blood vessels interferes with the movement of body fluids in and out of the various fluid compartments, so that fluid accumulates in the tissue spaces, causing edema.

The terms “edema” and “fluid retention” as used herein refer to the accumulation of excess fluid in a body compartment; the accumulation may be in the cells (cellular edema), in the intercellular spaces within tissues (interstitial edema), or in potential spaces within the body.

The term “heart failure” as used herein refers to the inability of the heart to maintain cardiac output sufficient to meet the body's needs; it most often results from myocardial failure affecting the right or left ventricle.

The term “PPAR” as used herein refers to peroxisome proliferator-activated receptor.

The terms “PPAR-gamma” and “PPAR-γ” as used herein refer to peroxisome proliferator-activated receptor gamma.

The term “PPAR-gamma agonist” as used herein refers to any agent that elicits a ten percent (10%) or greater increase in activity of the PPAR-gamma transcription factor, irrespective of the agent's mechanism, and without differentiation of full agonists from partial agonists, modulators, and allosteric modulators or allosteric agonists. In some embodiments, the PPAR-gamma agonist is any agent that causes a ten percent (10%) or greater induction (relative to vehicle) of PPAR-gamma transcriptional activity as measured in a suitable reporter gene assay. Examples of suitable reporter gene assays are described in Reginato M J et al., J Biol Chem 273:32679-84, 1998; and Schupp M, et al., Circulation 109:2054-7, 2004.

### DETAILED EMBODIMENTS

In one aspect, the present invention provides methods for reducing of fluid retention in a subject, comprising administering to a subject in need thereof a therapeutically effective amount of a sodium-dependent glucose transporter 2 inhibitor (SGLT2 inhibitor). In some embodiments, the fluid retention is associated with administration of a PPAR-gamma agonist to the subject. While PPAR-gamma agonists can have significant utility in the treatment of disease such of Type 2 diabetes, they are often accompanied by the undesirable side effect of edema, thereby limiting their utility. Accordingly, the present invention provides methods for expanding that the use of such PPAR-gamma agonists, when used in combination with an SGLT2 inhibitor. In selected embodiments, the PPAR-gamma agonist is a member selected from the group consisting of rosiglitazone, pioglitazone, rivoglitazone, netoglitazone and THR-0921 (formerly known as CLX-0921, described in Dey D, et al., Metabolism 52:1012-8, 2003). In other selected embodiments, the PPAR-gamma agonist is a member selected from the group consisting of aleglitazar, farglitazar, tesaglitazar, naveglitazar and muraglitazar. In other selected embodiments the PPAR-gamma agonist is a modulator selected from the group consisting of MBX-102 (Chandalia A, et al., PPAR Res 2009: article ID 706852, 12 pages, 2009), MBX-2044 (Metabolex, Inc.), PAR-1622 (Kim M K, et al., Arch Pharm Res 32:721-7, 2009) and INT131 (Higgins L S, et al., PPAR Res 2008: article ID 936906, 9 pages, 2008; Motani A, et al., J Mol Biol 386:1301-11, 2009).

In still other embodiments, the subject is at risk for development of a disease or condition associated with fluid retention. In one group of embodiments, the disease or condition is congestive heart failure. More particularly, the subject is a human with congestive heart failure. In one group of embodiments, the congestive heart failure is New York Heart Association (NYHA) Class I or II heart failure. In another group of embodiments, the congestive heart failure is NYHA Class III or IV heart failure.

In yet another group of embodiments, the method is carried out with further administration of an effective amount of a diuretic. Suitable diuretics are selected from loop diuretics and thiazide and thiazide-like diuretics. Such suitable diuretics are described, for example, in Goodman and Gilman's The Pharmacological Basis of Therapeutics, 11^{th} Ed., Brunton, Lazo and Parker, Eds., McGraw-Hill (2006), which is hereby incorporated herein by reference.

In another aspect, the present invention provides a method for the treatment of type 2 diabetes, comprising administering to a subject in need thereof, a therapeutically effective amount of a combination of one or more SGLT2 inhibitors and one or more PPAR-gamma agonists, wherein said subject is at risk for development of a disease or condition associated with fluid retention. In one group of embodiments, the disease or condition is congestive heart failure. As above, a variety of PPAR-gamma agonists are useful in this invention, including full and partial agonists, which may be selective or non-selective to PPAR-gamma (relative to alpha and delta isoforms). In one group of embodiments, the PPAR-gamma agonist is a member selected from the group consisting of rosiglitazone, pioglitazone, rivoglitazone, netoglitazone and THR-0921. Similarly, suitable PPAR-gamma agonists include, but are not limited to, MBX-102, MBX-2044, PAR-1622, INT131, muraglitazar, tesaglitazar, naveglitazar, aleglitazar and farglitazar.

In one group of embodiments, the method of the invention is carried out with further administering of an effective amount of a diuretic. Suitable diuretics can be selected from loop diuretics and thiazide and thiazide-like diuretics.

In still another aspect, the present invention provides a method for the treatment of type 2 diabetes, comprising administering to a subject in need thereof, a therapeutically effective amount of a combination of an SGLT2 inhibitor and a PPAR-gamma agonist, wherein said subject has a disease or condition associated with fluid retention. In some embodiments, the disease or condition is heart failure, which can be characterized as NYHA Class I or II heart failure, or can be characterized as NYHA Class III or IV heart failure. In general, suitable PPAR-gamma agonists are those that have been described above (e.g., the glitazones and glitazars). In one group of embodiments, the PPAR-gamma agonist is selected from the group consisting of rosiglitazone, pioglitazone, netoglitazone, rivoglitazone and THR-0921. In another group of embodiments, the PPAR-gamma agonist is selected from the group consisting of muraglitazar, tesaglitazar, naveglitazar, aleglitazar and farglitazar. In still another group of embodiments, the PPAR-gamma agonist is selected from the group consisting of INT131, MBX-102, MBX-2044 and PAR-1622. As with the methods and treatments above, this aspect of the invention can also include administering an effective amount of a diuretic. Suitable diuretics are selected from the group consisting of loop diuretics and thiazide and thiazide-like diuretics.

The present invention also provides methods of using one or more SGLT2 inhibitors for the preparation of a medicament for treating fluid retention or edema.

In another aspect, the invention provides pharmaceutical compositions comprising a mixture of therapeutically effective amounts of one or more SGLT2 inhibitors and one or more PPAR-gamma agonists. In a further embodiment, the invention provides pharmaceutical compositions consisting essentially of therapeutically effective amounts of an SGLT2 inhibitor and a PPAR-gamma agonist.

### Conditions Subject to Treatment

The present invention is useful for reducing edema or fluid retention in a subject in need thereof. The current invention is also unseful for the treatment of type 2 diabetes in a subject at risk for development of a disease or condition associated with fluid retention. Subjects at risk for development of a disease or condition associated with fluid retention include, for example, individuals with occluded coronary arteries, individuals who have experienced myocardial infarction, and overweight or obese individuals (i.e., individuals having a body mass index greater than 25). Furthermore, the present invention is useful for the treatment of type 2 diabetes in a subject having a disease or condition associated with fluid retention. Diseases and conditions associated with fluid retention include, for example, heart failure, particularly congestive heart failure, and pulmonary hypertension.

### Pharmacological Agents

In any of the methods described above, a variety of SGLT2 inhibitors can be used, typically selected from Formulae I, II, III, IV or V below.

Accordingly, in one embodiment, the SGLT2 inhibitors for use in the present invention are compounds of Formula I:

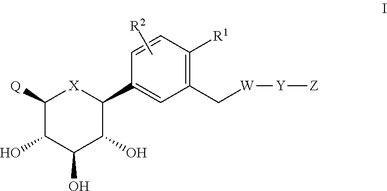

wherein

X represents oxygen or sulfur;

Q represents —CH₂OH, C₁-C₆ alkylsulfanyl, C₁-C₆ alkylsulfinyl, C₁-C₆ alkylsulfonyl, C₁-C₆ haloalkylsulfanyl, C₁-C₆ haloalkylsulfinyl, C₁-C₆ haloalkylsulfonyl, or —CH₂OV, where V represents (C₁-C₃ alkyl)oxycarbonyl, (C₁-C₆ alkyl)carbonyl, phenyloxycarbonyl, benzylcarbonyl or benzyloxycarbonyl;

R¹ and R² each independently represent hydrogen, halo, C₁-C₃ alkyl, C₂-C₃ alkynyl, C₃-C₆ cycloalkyl, hydroxy or cyano;

W represents a 5- to 6-membered aryl or heteroaryl ring, or an 8- to 10-membered fused bicyclic aryl or heteroaryl ring,

wherein W optionally may be mono- or disubstituted by identical or different substituents selected from halo, hydroxy, C₁-C₃ alkyl, C₁-C₃ alkoxy, cyano, —NR^{a}R^{b}, —C(O)NR^{a}R^{b}, C₁-C₆ alkylsulfanyl, C₁-C₆ alkylsulfinyl, and C₁-C₆ alkylsulfonyl, and
wherein alkyl groups or portions optionally may be partly or completely fluorinated;

Y represents a single bond or a 5- to 6-membered aryl or heteroaryl ring,

wherein Y optionally may be mono- or disubstituted by identical or different substituents selected from halo, hydroxy, C₁-C₃ alkyl, C₁-C₃ alkoxy, cyano, —NR^{a}R^{b}, —C(O)NR^{a}R^{b}, C₁-C₆ alkylsulfanyl, C₁-C₆ alkylsulfinyl, and C₁-C₆ alkylsulfonyl, and
wherein alkyl groups or portions optionally may be partly or completely fluorinated;

Z represents hydrogen, halo, hydroxy, cyano, C₁-C₃ alkyl, C₁-C₃ alkoxy, C₂-C₃ alkynyl, C₃-C₆ cycloalkyl, C₃-C₆ heterocycloalkyl, C₃-C₆ cycloalkoxy, C₃-C₆ heterocycloalkoxy, (C₁-C₃ alkoxy)C₁-C₃ alkoxy or (C₃-C₆ cycloalkoxy)C₁-C₃ alkoxy,

wherein alkyl, alkynyl, cycloalkyl and heterocycloalkyl groups or portions optionally may be partly or completely fluorinated and may be mono- or disubstituted by identical or different substituents selected from chloro, hydroxy, C₁-C₃ alkyl, C₁-C₃ alkoxy, cyano, —NR^{a}R^{b}, —C(O)NR^{a}R^{b}, C₁-C₆ alkylsulfanyl, C₁-C₆ alkylsulfinyl, and C₁-C₆ alkylsulfonyl;

R^{a} and R^{b} each independently represent hydrogen or C₁-C₆ alkyl, wherein alkyl groups optionally may be partly or completely fluorinated; and

wherein optionally one or more hydrogen atoms may be substituted with deuterium.

In certain preferred embodiments of compounds of Formula I for use in the invention, X represents oxygen or sulfur; Q represents —CH₂OH or methylsulfonyl; R¹ represents hydrogen, chloro, fluoro, methyl or cyano; R² represents hydrogen or hydroxy; W represents phenyl; Y represents a single bond; and Z represents ethyl, ethoxy, ethynyl, cyclopropyl, benzo[b]thiophen-2-yl, azulenyl, tetrahydrofuran-3-yloxy or cyclopropoxyethoxy.

In particularly preferred embodiments, compounds of Formula I for use in the present invention are selected from:

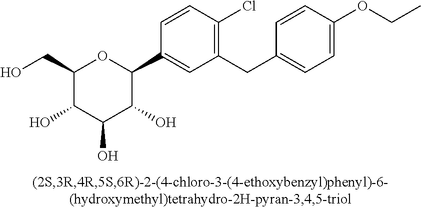

which is described in WO 03/099836, with crystal forms described in WO 2008/002824;

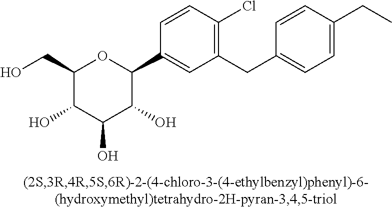

which is described in WO 2006/034489;

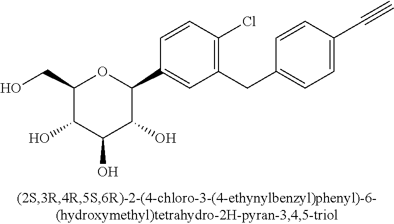

which is described in US 2005/0209166, with crystal forms described in US 2007/0054867;

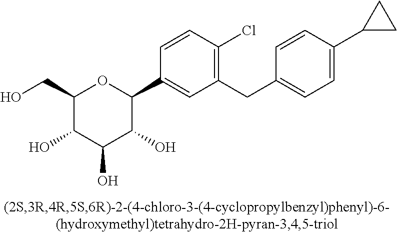

which is described in CN 200810176680.7 and U.S. 61/134,968;

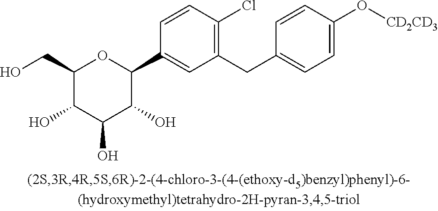

which is described in U.S. 61/134,968;

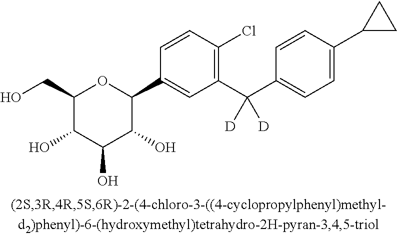

which is described in U.S. 61/134,968;

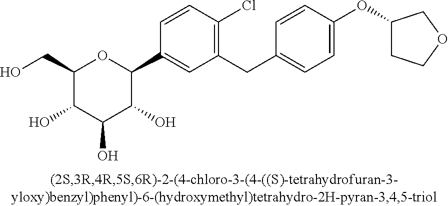

which is described in US 2005/0209166, with crystal forms described in WO 2006/117359;

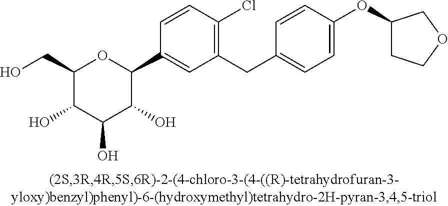

which is described in US 2005/0209166, with crystal forms described in WO 2006/117360;

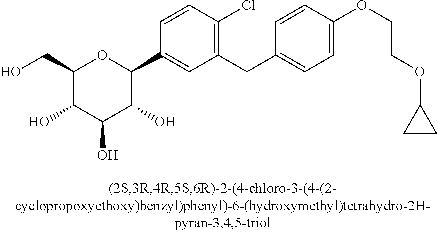

which is described along with an amino acid co-crystal in US 2009/0118201;

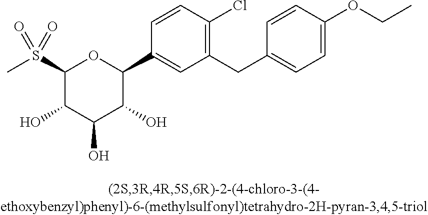

which is described in US 2008/0113922;

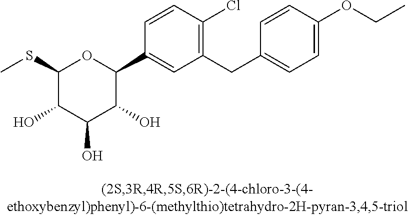

which is described in US 2008/0113922, with crystal forms described in US 2009/0030198;

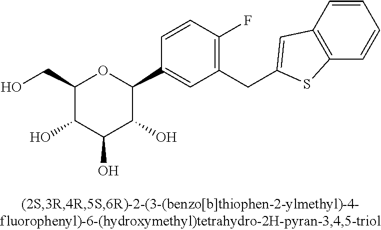

which is described in EP 1 609 785 and WO 2005/012326, with crystal forms described in EP 2 009 010;

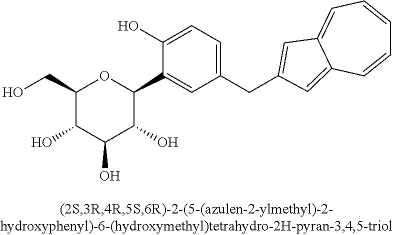

which is described in EP 1 553 094 and EP 1 783 122, with crystal forms described in EP 1 908 757;

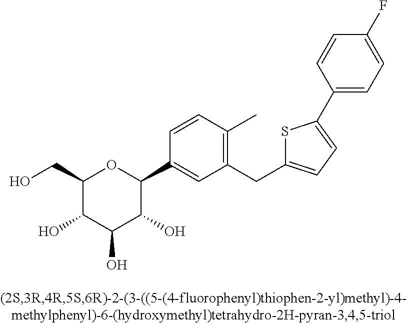

which is described in WO 2005/012326, with crystalline forms described in WO 2008/069327 and WO 2009/035969;

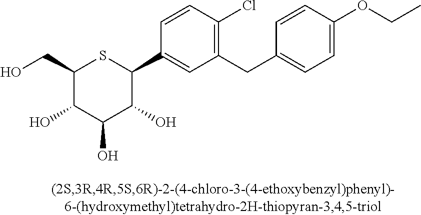

which is described in US 2008/0132563;

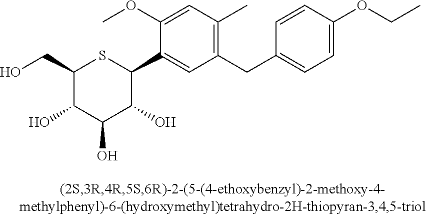

which is described in US 2008/0132563; and

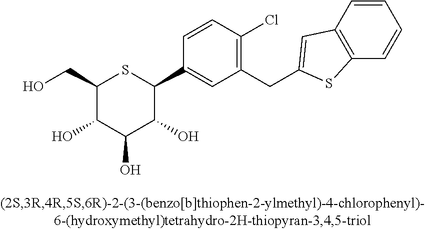

which is described in US 2008/0132563.

In another aspect, the SGLT2 inhibitors for use in the present invention are compounds of Formula II:

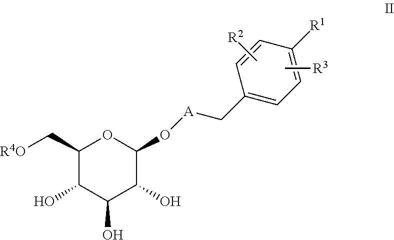

wherein

A represents a 5- to 6-membered aryl or heteroaryl ring,

wherein A optionally may be mono- or disubstituted by identical or different substituents selected from halo, hydroxy and C₁-C₆ alkyl, and
wherein alkyl groups or portions optionally may be partly or completely fluorinated;

R¹ represents C₁-C₃ alkoxy, wherein the alkyl portion optionally may be partly or completely fluorinated;

R² and R³ each independently represent hydrogen, halo or C₁-C₃ alkyl, wherein the alkyl group optionally may be partly or completely fluorinated; and

R⁴ represents hydrogen, (C₁₋₆ alkyl)carbonyl, (C₁₋₃ alkyl)oxycarbonyl, phenyloxycarbonyl, benzyloxycarbonyl or benzylcarbonyl.

In certain preferred embodiments of compounds of Formula II for use in the invention, A represents benzene, tetrahydrothiophene or 1-isopropyl-5-methyl-1H-pyrazole; R¹ represents methoxy, trifluoromethoxy or isopropoxy; R² and R³ each represent hydrogen; and R⁴ represents hydrogen or ethoxycarbonyl.

In particularly preferred embodiments, compounds of Formula II for use in the present invention are selected from:

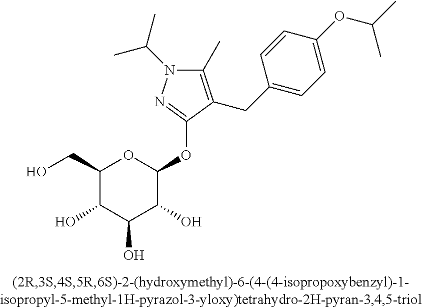

which is described in EP 1 354 888;

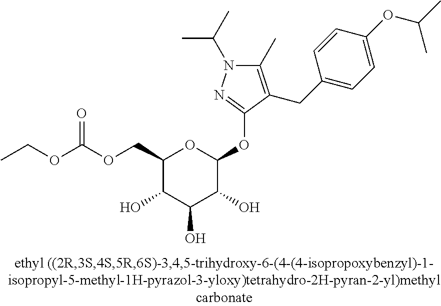

which is described in EP 1 354 888;

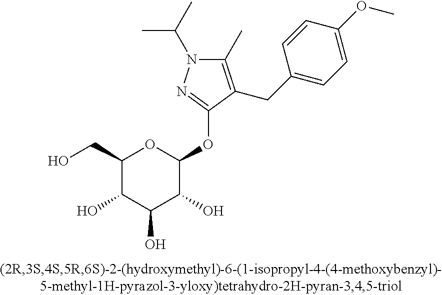

which is described in EP 1 338 603, with crystal forms described in US2007/0244176;

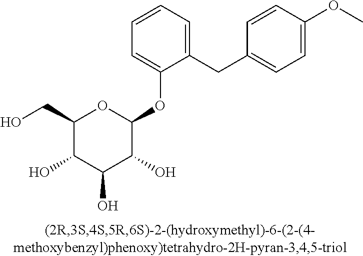

which is described in EP 1 329 456;

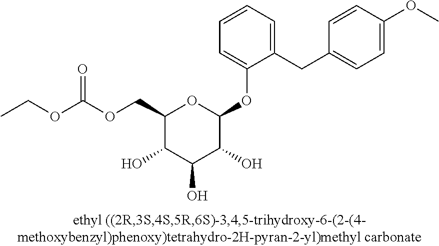

which is described in EP 1 329 456, with crystal forms described in EP 1 489 089;

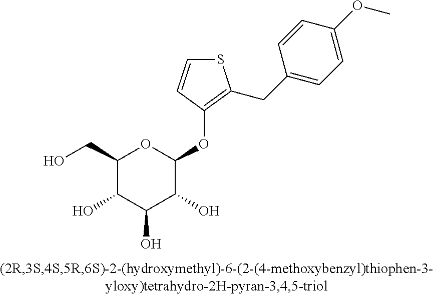

which is described in US 2004/0138143 and US 2008/0207882; and

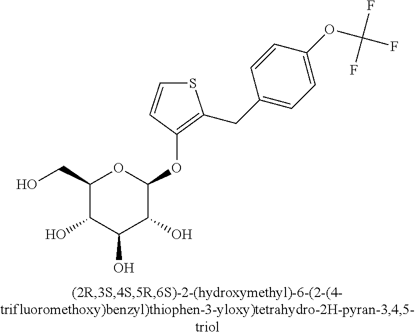

which is described in US 2004/0138143 and US 2008/0207882.

In another aspect, the SGLT2 inhibitors for use in the present invention are compounds of Formula III:

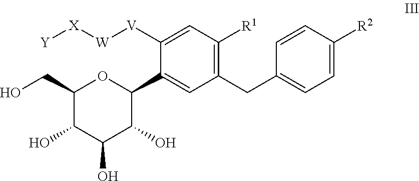

wherein

V represents oxygen or a single bond;

W represents C₁-C₆ alkylene;

X represents oxygen or sulfur;

Y represents C₁-C₆ haloalkyl, C₁-C₆ hydroxyalkyl, C₂-C₆ alkenyl, C₂-C₆ alkynyl, C₃-C₁₀ cycloalkyl, C₅-C₁₀ cycloalkenyl, (C₁-C₄ alkoxy)C₁-C₃ alkyl, (C₂-C₄ alkenyloxy)C₁-C₃ alkyl or (C₃-C₁₀ cycloalkyloxy)C₁-C₃ alkyl;

wherein alkyl, alkenyl, alkynyl, cycloalkyl and cycloalkenyl groups or portions optionally may be partly or completely fluorinated and may be mono- or disubstituted by identical or different substituents selected from chlorine, hydroxy, C₁-C₃ alkyl and C₁-C₃ alkoxy;

R¹ represents hydrogen, halo, cyano, C₁-C₆ alkyl or C₃-C₁₀ cycloalkyl; and

R² represents hydrogen, halo, hydroxy, C₁-C₆ alkyl, C₁-C₆ alkyloxy, C₂-C₆ alkenyl, C₂-C₆ alkynyl, C₃-C₁₀ cycloalkyl, or C₃-C₁₀ cycloalkoxy, wherein alkyl and cycloalkyl groups or portions optionally may be partly or completely fluorinated.

In certain preferred embodiments of compounds of Formula III for use in the invention, V represents oxygen or a single bond; W represents C₁-C₃ alkylene; X represents oxygen; Y represents C₁-C₃ haloalkyl, C₂-C₄ alkenyl or C₂-C₄ alkynyl; R¹ represents halo; and R² represents C₁-C₃ alkyl or C₁-C₃ alkoxy.

In particularly preferred embodiments, compounds of Formula III for use in the present invention are selected from:

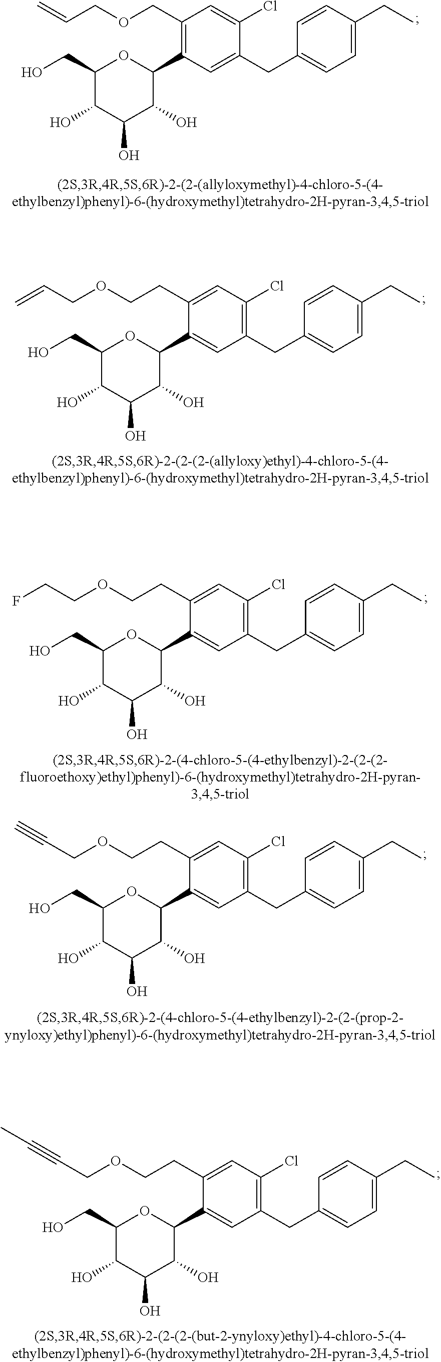

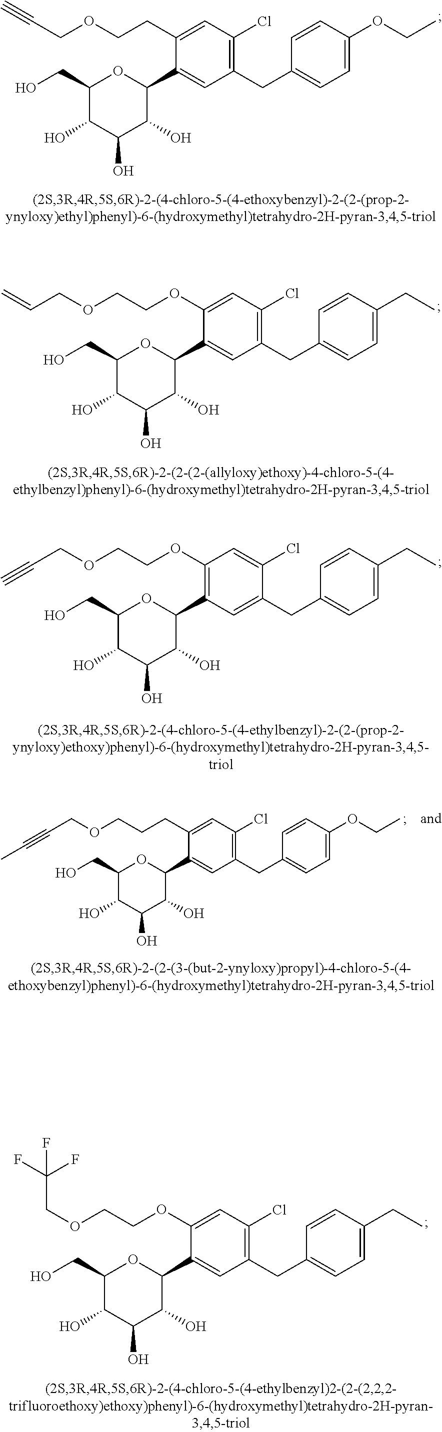

which are described in US 2008/0242596.

In another aspect, the SGLT2 inhibitors for use in the present invention are compounds of Formula IV:

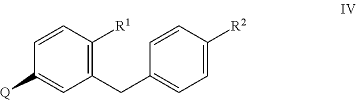

wherein

R¹ represents hydrogen, halo, C₁-C₃ alkyl or C₁-C₃ alkoxy; R² represents C₁-C₃ alkyl, C₂-C₄ alkenyl, C₂-C₄ alkynyl, C₃-C₆ cycloalkyl or C₁-C₃ alkoxy; and Q is selected from the following formulae Q^{1A} to Q^{4A}:

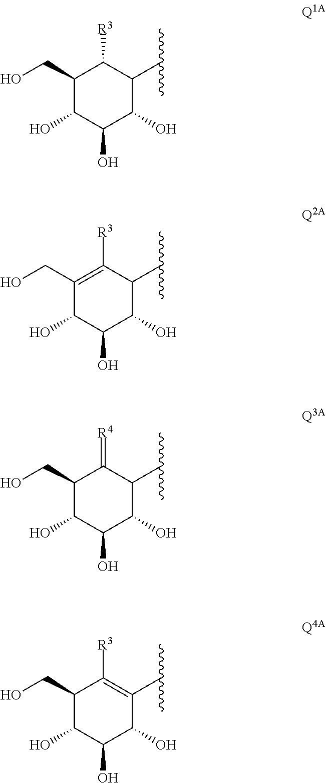

wherein R³ represents hydrogen or hydroxy, and R⁴ represents oxygen or CR^{a}R^{b}, wherein R^{a} and R^{b} each independently represent hydrogen or halo.

In certain preferred embodiments of compounds of Formula IV for use in the invention, R¹ represents hydrogen or halo; R² represents C₁-C₃ alkyl or C₁-C₃ alkoxy; R³ represents hydrogen or hydroxy; and R⁴ represents oxygen.

In particularly preferred embodiments, compounds of Formula IV for use in the present invention are selected from:

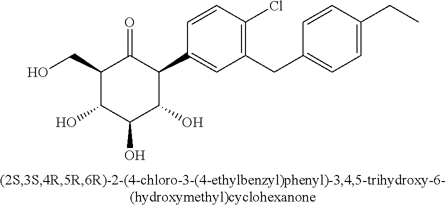

which is described in WO 2009/076550;

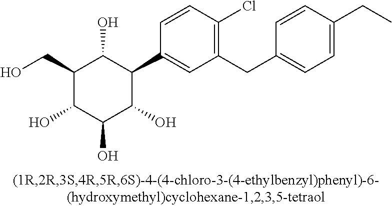

which is described in WO 2009/076550; and

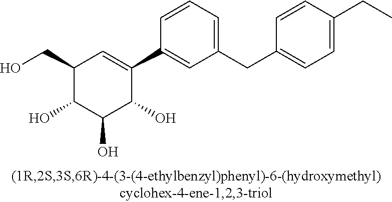

which is described in EP 1 783 110.

In another aspect, the SGLT2 inhibitors for use in the present invention are compounds of Formula V:

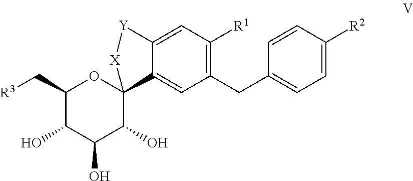

wherein

X represents methylene or oxygen; Y represents (CH₂)ₙ, (CH₂)ₘCH═CH, CH═CH(CH₂)ₘ, or CH₂CH═CHCH₂, wherein n is an integer from 1 to 3 and m is an integer from 0 to 2; R¹ represents hydrogen or halo; R² represents hydrogen, halo, C₁-C₃ alkyl, C₂-C₄ alkynyl, C₃-C₆ cycloalkyl, C₁-C₃ alkoxy, C₃-C₆ cycloalkoxy, hydroxy or cyano; R³ represents hydroxy, fluoro or C₁-C₃ alkoxy; and wherein alkyl groups or portions optionally may be partly or completely fluorinated.

In certain preferred embodiments of compounds of Formula V for use in the invention, X represents oxygen; Y represents CH₂; R¹ represents hydrogen or halo; R² represents C₁-C₃ alkyl or C₁-C₃ alkoxy, wherein the alkyl group or portion optionally may be partly or completely fluorinated; and R³ represents hydroxy.

In other preferred embodiments of compounds of Formula V for use in the invention, X represents methylene; Y represents CH₂; R¹ represents hydrogen or halo; R² represents C₁-C₃ alkyl or C₁-C₃ alkoxy, wherein the alkyl group or portion optionally may be partly or completely fluorinated; and R³ represents hydroxy.

In particularly preferred embodiments, compounds of Formula V for use in the present invention are selected from:

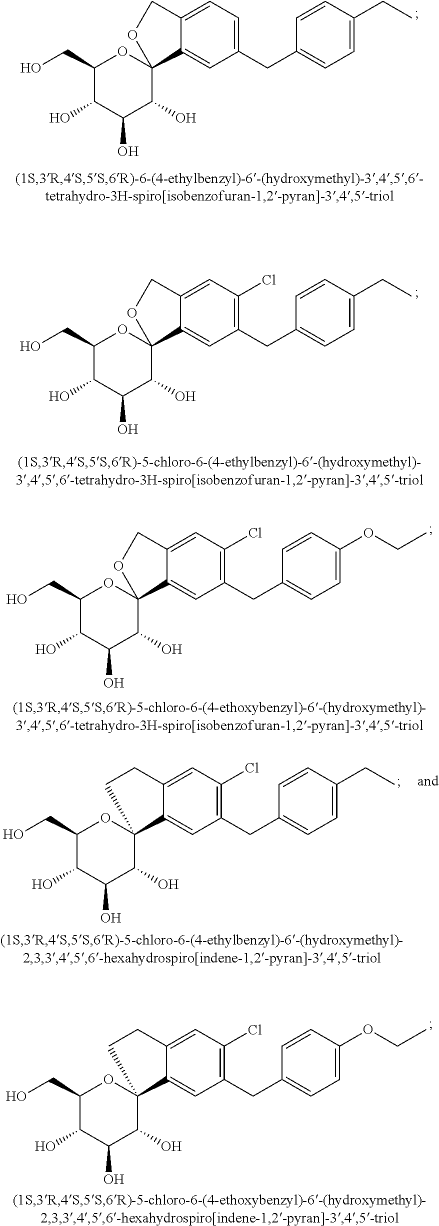

which are described in US 2007/0275907.

The style used above and hereinafter, in which a bond of a substituent on a phenyl group is shown ending near the center of the phenyl ring, denotes, unless otherwise stated, that this substituent may be bound to any free position of the phenyl group bearing a hydrogen atom. The names of compounds were derived from the structures shown using the CambridgeSoft Struct=Name algorithm as implemented in ChemDraw Ultra version 10.0.

The present invention includes the use of all tautomers and stereoisomers of the afore-mentioned compounds, either in admixture or in pure or substantially pure form. The compounds can have asymmetric centers at the carbon atoms, and therefore the compounds can exist in diastereomeric or enantiomeric forms or mixtures thereof. The use of all conformational isomers (e.g., cis and trans isomers) and all optical isomers (e.g., enantiomers and diastereomers), racemic, diastereomeric and other mixtures of such isomers, as well as solvates, hydrates, isomorphs, polymorphs and tautomers are within the scope of the present invention. The compounds can be prepared using diastereomers, enantiomers or racemic mixtures as starting materials. Furthermore, diastereomer and enantiomer products can be separated by chromatography, fractional crystallization or other methods known to those of skill in the art.

The present invention also provides for the use of prodrugs of the afore-mentioned compounds. Prodrugs of the compounds include, but are not limited to, carboxylate esters, carbonate esters, hemi-esters, phosphorus esters, nitro esters, sulfate esters, sulfoxides, amides, carbamates, azo compounds, phosphamides, glycosides, ethers, acetals, and ketals. Prodrug esters and carbonates may be formed, for example, by reacting one or more hydroxyl groups of the compounds with alkyl, alkoxy or aryl substituted acylating reagents using methods known to those of skill in the art to produce methyl carbonates, acetates, benzoates, pivalates and the like. Illustrative examples of prodrug esters of the compounds include, but are not limited to, compounds having a hydroxy moiety wherein the free hydrogen is replaced by (C₁-C₆ alkyl)oxycarbonyl, (C₁-C₆ alkyl)carbonyl, phenyloxycarbonyl, benzylcarbonyl or benzyloxycarbonyl. The use of oligopeptide modifications and biodegradable polymer derivatives (as described, for example, in Int. J. Pharm. 115, 61-67, 1995) are within the scope of the invention. Methods for selecting and preparing suitable prodrugs are provided, for example, in the following: T. Higuchi and V. Stella, “Prodrugs as Novel Delivery Systems,” Vol. 14, ACS Symposium Series, 1975; H. Bundgaard, “Design of Prodrugs,” Elsevier, 1985; and “Bioreversible Carriers in Drug Design,” ed. Edward Roche, American Pharmaceutical Association and Pergamon Press, 1987.

The present invention also provides for the use of the pharmaceutically acceptable salts of the afore-mentioned compounds and prodrugs thereof. The acids that can be used as reagents to prepare the pharmaceutically acceptable acid addition salts of the basic compounds for use in this invention are those which form non-toxic acid addition salts, i.e., salts containing pharmacologically acceptable anions (such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate (1,1′-methylene-bis-2-hydroxy-3-naphthoate) salts). The bases that can be used as reagents to prepare the pharmaceutically acceptable base salts of the acidic compounds for use in the present invention are those that form non-toxic base salts with such compounds, including, but not limited to, those derived from pharmacologically acceptable cations such as alkali metal cations (e.g., potassium, lithium and sodium) and alkaline earth metal cations (e.g., calcium and magnesium), ammonium or water-soluble amine addition salts such as N-methylglucamine (meglumine), and the lower alkanolammonium and other base salts of pharmaceutically acceptable organic amines (e.g., methylamine, ethylamine, propyl amine, dimethyl amine, triethanolamine, diethylamine, t-butylamine, t-octylamine, trimethylamine, triethylamine, ethylenediamine, hydroxyethylamine, morpholine, piperazine, dehydroabietylamine, lysine and guanidine).

The present invention also includes the use of isotopically-labeled compounds, wherein one or more atoms are replaced by one or more atoms having specific atomic mass or mass numbers. Examples of isotopes that can be incorporated into compounds for use in the invention include, but are not limited to, isotopes of hydrogen, carbon, nitrogen, oxygen, fluorine, sulfur, and chlorine (such as ²H, ³H, ¹³C, ¹⁴C, ¹⁵N, ¹⁸O, ¹⁷O, ¹⁸F, ³⁵S and ³⁶Cl). The use of isotopically-labeled compounds and prodrugs thereof, as well as isotopically-labeled, pharmaceutically acceptable salts of compounds and prodrugs thereof, are within the scope of the present invention. For example, in certain circumstances substitution with heavier isotopes, such as deuterium (²H), can provide increased metabolic stability, which offers therapeutic advantages such as increased in vivo half-life or reduced dosage requirements. Any of the chemical groups, functional groups, or substituents described herein may be deuterated if the chemical group, functional group, or substituent has —H. Isotopically-labeled compounds and prodrugs thereof can generally be prepared according to the methods described in the references cited herein by substituting an isotopically-labeled reagent for a non-isotopically labeled reagent.

SGLT2 inhibitors for use in the present invention may be synthesized by the methods described in the references cited above and by techniques generally known to those skilled in the art without undue experimentation.

Optionally, the compounds may be reacted with a complex forming reagent, such as the D or L enantiomer of a natural amino acid, in a suitable solvent to form the corresponding crystalline complex, such as the amino acid complex, of the compound. Amino acid complexes of compounds for use in the present invention may be formed by mixing an amino acid with the purified compound in a suitable solvent or with a crude reaction mixture containing the compound and other reagents.

### Pharmaceutical Compositions and Methods of Use

A compound for use in this invention can be incorporated into a variety of formulations for therapeutic administration. More particularly, a compound for use in the present invention can be formulated into pharmaceutical compositions, together or separately, by formulation with appropriate pharmaceutically acceptable carriers or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of a compound according to the present invention can be achieved in various ways, including oral, buccal, parenteral, intravenous, intradermal (e.g., subcutaneous, intramuscular), transdermal, etc., administration. Moreover, the compound can be administered in a local rather than systemic manner, for example, in a depot or sustained release formulation.

Suitable formulations for use in the present invention are found in Remington: The Science and Practice of Pharmacy, 21^{st} Ed., Gennaro, Ed., Lippencott Williams & Wilkins (2003), which is hereby incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

In one preferred embodiment, one or more SGLT2 inhibitors, independently or in combination with one or more PPAR-gamma agonists, is prepared for delivery in a sustained-release, controlled release, extended-release, timed-release or delayed-release formulation, for example, in semipermeable matrices of solid hydrophobic polymers containing the therapeutic agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients (see, for example, Huang, et al. Drug Dev. Ind. Pharm. 29:79 (2003); Pearnchob, et al. Drug Dev. Ind. Pharm. 29:925 (2003); Maggi, et al. Eur. J. Pharm. Biopharm. 55:99 (2003); Khanvilkar, et al., Drug Dev. Ind. Pharm. 228:601 (2002); and Schmidt, et al., Int. J. Pharm. 216:9 (2001)). Sustained-release delivery systems can, depending on their design, release the compounds over the course of hours or days, for instance, over 4, 6, 8, 10, 12, 16, 20, 24 hours or more. Usually, sustained release formulations can be prepared using naturally-occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

The sustained or extended-release formulations can also be prepared using natural ingredients, such as minerals, including titanium dioxide, silicon dioxide, zinc oxide, and clay (see, U.S. Pat. No. 6,638,521, herein incorporated by reference). Exemplified extended release formulations that can be used in delivering a compound of the present invention include those described in U.S. Pat. Nos. 6,635,680; 6,624,200; 6,613,361; 6,613,358, 6,596,308; 6,589,563; 6,562,375; 6,548,084; 6,541,020; 6,537,579; 6,528,080 and 6,524,621, each of which is hereby incorporated herein by reference. Controlled release formulations of particular interest include those described in U.S. Pat. Nos. 6,607,751; 6,599,529; 6,569,463; 6,565,883; 6,482,440; 6,403,597; 6,319,919; 6,150,354; 6,080,736; 5,672,356; 5,472,704; 5,445,829; 5,312,817 and 5,296,483, each of which is hereby incorporated herein by reference. Those skilled in the art will readily recognize other applicable sustained release formulations.

For oral administration, a compound for use in the present invention can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

The compounds can be formulated for parenteral administration by injection, e.g., by bolus injection or continuous infusion. For injection, the compound can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, a compound for use in the invention can be formulated in aqueous solutions, preferably in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

Systemic administration can also be by transmucosal or transdermal means. For transmucosal or transdermal administration, penetrants appropriate to the barrier to be permeated are used in the formulation. For topical administration, the agents are formulated into ointments, creams, salves, powders and gels. In one embodiment, the transdermal delivery agent can be DMSO. Transdermal delivery systems can include, e.g., patches. For transmucosal administration, penetrants appropriate to the barrier to be permeated are used in the formulation. Such penetrants are generally known in the art. Exemplified transdermal delivery formulations that can find use in the present invention include those described in U.S. Pat. Nos. 6,589,549; 6,544,548; 6,517,864; 6,512,010; 6,465,006; 6,379,696; 6,312,717 and 6,310,177, each of which are hereby incorporated herein by reference.

For buccal administration, the compositions can take the form of tablets or lozenges formulated in conventional manner.

In addition to the formulations described previously, a compound for use in the present invention can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions suitable for use in the present invention include compositions wherein the active ingredients are contained in a therapeutically effective amount. The present invention also contemplates pharmaceutical compositions comprising therapeutically effective amounts of one or more SGLT2 inhibitors in admixture with an effective amount of one or more PPAR-gamma agonists as combination partners. An effective amount of the compound and/or combination partner will, of course, be dependent on the subject being treated, the severity of the affliction and the manner of administration. Determination of an effective amount is well within the capability of those skilled in the art, especially in light of the detailed disclosure provided herein. Generally, an efficacious or effective amount of a compound is determined by first administering a low dose or small amount, and then incrementally increasing the administered dose or dosages until a desired therapeutic effect is observed in the treated subject, with minimal or no toxic side effects. Applicable methods for determining an appropriate dose and dosing schedule for administration of the present invention are described, for example, in Goodman and Gilman's The Pharmacological Basis of Therapeutics, 11^{th} Ed., Brunton, Lazo and Parker, Eds., McGraw-Hill (2006), and in Remington: The Science and Practice of Pharmacy, 21^{st} Ed., Gennaro, Ed., Lippencott Williams & Wilkins (2003), both of which are hereby incorporated herein by reference.

Furthermore, in another aspect, the invention provides for a pharmaceutical composition comprising effective amounts of one or more SGLT2 inhibitors, or a pharmaceutically acceptable salt or prodrug thereof, and at least one member selected from the group of PPAR-gamma agonists as combination partners, in a pharmaceutically acceptable carrier.

The treatment of the present invention can be administered prophylactically to prevent or delay the onset or progression of fluid retention or edema, or therapeutically to reduce the level of fluid retention in a subject, for example a subject undergoing treatment with a PPAR-gamma agonist which otherwise promotes fluid retention.

The SGLT2 inhibitors can be administered to a subject, e.g., a human patient, a domestic animal such as a cat or a dog, independently or together with a combination partner, in the form of their pharmaceutically acceptable salts or prodrugs, or in the form of a pharmaceutical composition where the SGLT2 inhibitors and/or combination partners are mixed with suitable carriers or excipient(s) in a therapeutically effective amount. Consequently, one or more SGLT2 inhibitors, or a pharmaceutically acceptable salt or prodrug thereof, and a PPAR-gamma agonist to be combined therewith, can be present in a single formulation, for example a capsule or tablet, or in two separate formulations, which can be the same or different, for example, in the form of a kit comprising selected numbers of doses of each agent.

The appropriate dosage of compound will vary according to the chosen route of administration and formulation of the composition, among other factors, such as patient response. The dosage can be increased or decreased over time, as required by an individual patient. A patient initially may be given a low dose, which is then increased to an efficacious dosage tolerable to the patient. Typically, a useful dosage for adults may be from 1 to 2000 mg, preferably 1 to 200 mg, when administered by oral route, and from 0.1 to 100 mg, preferably 1 to 30 mg, when administered by intravenous route, in each case administered from 1 to 4 times per day.

Dosage amount and interval can be adjusted individually to provide plasma levels of the active compounds which are sufficient to maintain therapeutic effect. Preferably, therapeutically effective serum levels will be achieved by administering single daily doses, but efficacious multiple daily dose schedules are included in the invention. In cases of local administration or selective uptake, the effective local concentration of the drug may not be related to plasma concentration. One having skill in the art will be able to optimize therapeutically effective local dosages without undue experimentation.

All publications and patent applications cited in this specification are herein incorporated by reference as if each individual publication or patent application were specifically and individually indicated to be incorporated by reference. Any conflict between any reference cited herein and the teaching of this specification is to be resolved in favor of the latter. Similarly, any conflict between an art-recognized definition of a word or phrase and a definition of the word or phrase as provided in this specification is to be resolved in favor of the latter. Although the foregoing invention has been described in some detail by way of illustration and example for purposes of clarity of understanding, it will be readily apparent to those of ordinary skill in the art in light of the teachings of this invention that certain changes and modifications can be made thereto without departing from the spirit or scope of the appended claims. The invention will be described in greater detail by way of specific example.

### EXAMPLE

The following example is offered for illustrative purposes, and is not intended to limit the invention in any manner. Those of skill in the art will readily recognize a variety of noncritical parameters which can be changed or modified to yield essentially the same results.

### Example 1

This example illustrates the reduction in fluid retention in a mammal administered an SGLT2 inhibitor (“TEST COMPOUND: (2S,3R,4R,5S,6R)-2-(4-chloro-3-(4-(2-cyclopropoxyethoxy)benzyl)phenyl)-6-(hydroxymethyl)tetrahydro-2H-pyran-3,4,5-triol”) in combination with pioglitazone. Amiloride was also evaluated as a reference standard.

Materials

Test Substance, Reference Substance(s), and Vehicle Storage and Formulation of Test Substance

Name: TEST COMPOUND Appearance: White powder; Storage: Stored in amber glass bottles at room temperature, tightly closed. Keep away from light and humidity.

Formulation: Dose preparation was performed once for the entire week. Baseline group mean body weights and water consumption were used to calculate the appropriate target concentrations for each treatment group. The appropriate amount of the test article was accurately weighed out with a calibrated electrical balance into glass flasks and dissolved in tap water to achieve the desired final concentration.

Storage and Formulation of Reference Substance

Name: Amiloride HCl Hydrate (Sigma-Aldrich; St Louis, Mo.); Appearance: Yellow powder; Purity: >98%

Storage: Stored in amber glass bottles at room temperature, tightly closed. Keep away from light and humidity.

Formulation: Dose preparation was performed once for the entire week. Baseline group mean body weights and water consumption were used to calculate the appropriate target concentrations for each treatment group. The appropriate amount of the reference article was accurately weighed out with a calibrated electrical balance into glass flasks and dissolved in tap water to achieve the desired final concentration.

Vehicle
Name: Water

Methods

Study Animals
Animal Source and Strain: Sprague-Dawley rats; Age: 10 weeks old; Gender: Males; Source: Charles River Laboratories

Housing

The study was performed in an SPF level animal room at the Molecular Medicine Research Institute. The room illumination was maintained using an automatic timer set for a light/dark cycle of 12 hours on and 12 hours off (lights off: 6:00 pm) with no twilight. Animals were individually housed in plastic cages. Alpha-Dry was used as bedding and was changed once per week. Cages, tops, and bottles were washed with a commercial detergent and allowed to air dry. A commercial disinfectant was used to disinfect surfaces and materials introduced into the room. A cage card or label with the appropriate information necessary to identify the study, dose, animal number and treatment group was used to mark all cages. Temperature and relative humidity were recorded during the study, and were consistently maintained within recommended ranges (18-26° C. and 30-70%).

Treatment Groups

The following treatment groups were employed: 1) vehicle control, 2) 3.0 mg/kg of test article and 3) 1.0 mg/kg of reference article.

Study Design

On day −5 rats and water bottles were weighed and placed in the cage with ad lib access to standard rodent chow. Body weights were used to assign rats to counter-balanced groups. Daily water consumption was estimated after weighing bottles again on day −1 and used to determine the appropriate drug concentrations to achieve the desired daily doses.

On day 1, rats were weighed and then received an ad mixed diet (ad lib) containing 0.03% pioglitazone in place of standard chow. Food weight was recorded in order to track consumption over the course of the experiment. At this time, rats were also given drinking water containing compound at the appropriate concentration. Full bottles were pre-weighed and recorded in order to track consumption over the course of the experiment.

On day 1 and day 7, rats were warmed using a heating pad and lightly anesthetized using isoflurane (Butler Animal Health Services; Chicago, Ill.). Using a sterile scalpel blade, a small tail nick was made on the distal one third of the tail. Blood was collected in a heparinized microcapillary tube (FisherBrand; Pittsburg, Pa.), sealed with putty at one end and stored on ice until centrifugation (5000 rpm, 5 min). Plasma volume was determined using a Critocaps microhematocrit capillary tube reader card (Leica: Lot #7426).

On day 7, rats, food and water bottles were again weighed and recorded, hematocrits were determined using blood collected by a tail nick.

Statistical Analysis

All data are presented as means +/−1 SEM. Statistical analyses were performed using GraphPad Prism statistical software. The mean differences among the treatment groups for all variables were tested for statistical significance using 2-way repeated measures ANOVAs. Additionally, differences were calculated for each animal (day 7−day 1), and resultant means were tested for statistical significance using 1-way ANOVAs. P values less than 0.05 were considered to be statistically significant and were followed by pair-wise comparisons using Dunnett's correction (overall alpha ≦0.05).

The daily dose of pioglitazone received by each rat was calculated from the total food consumption and the animal's average body weight. Daily dose of TEST COMPOUND or amiloride received by each rat was calculated from the total water consumption and the animal's average body weight.

### Results

### Plasma Volume (Hematocrit):

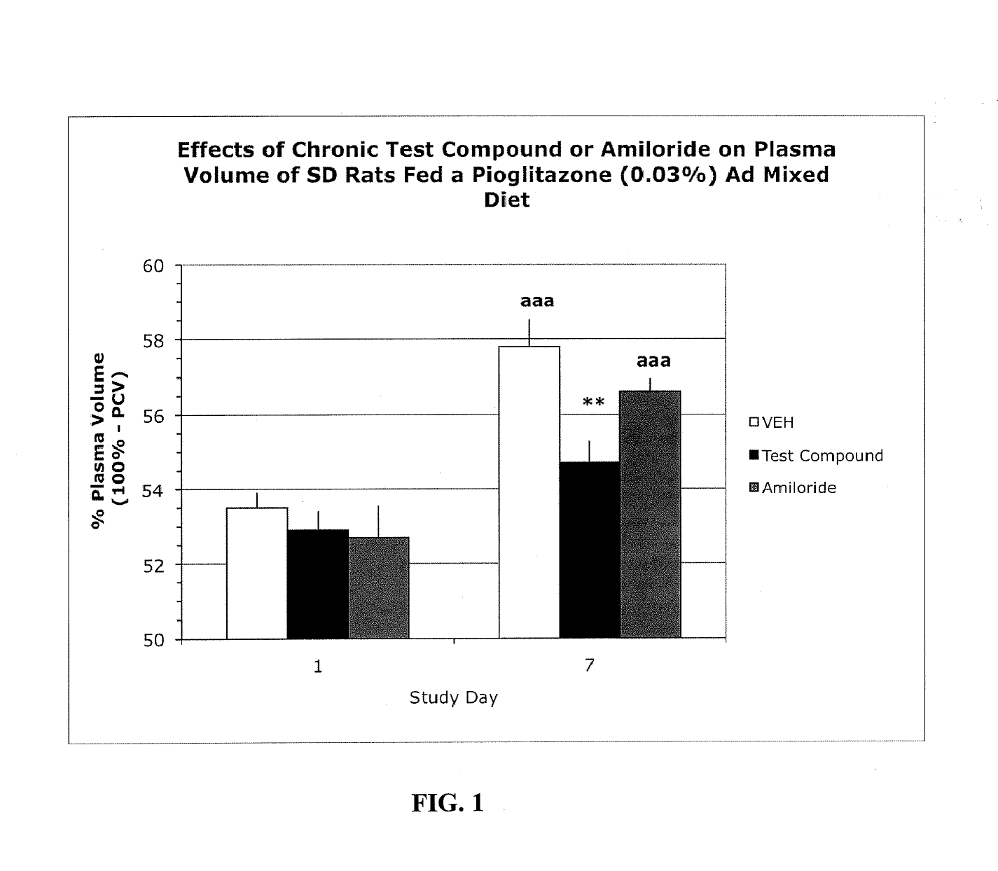

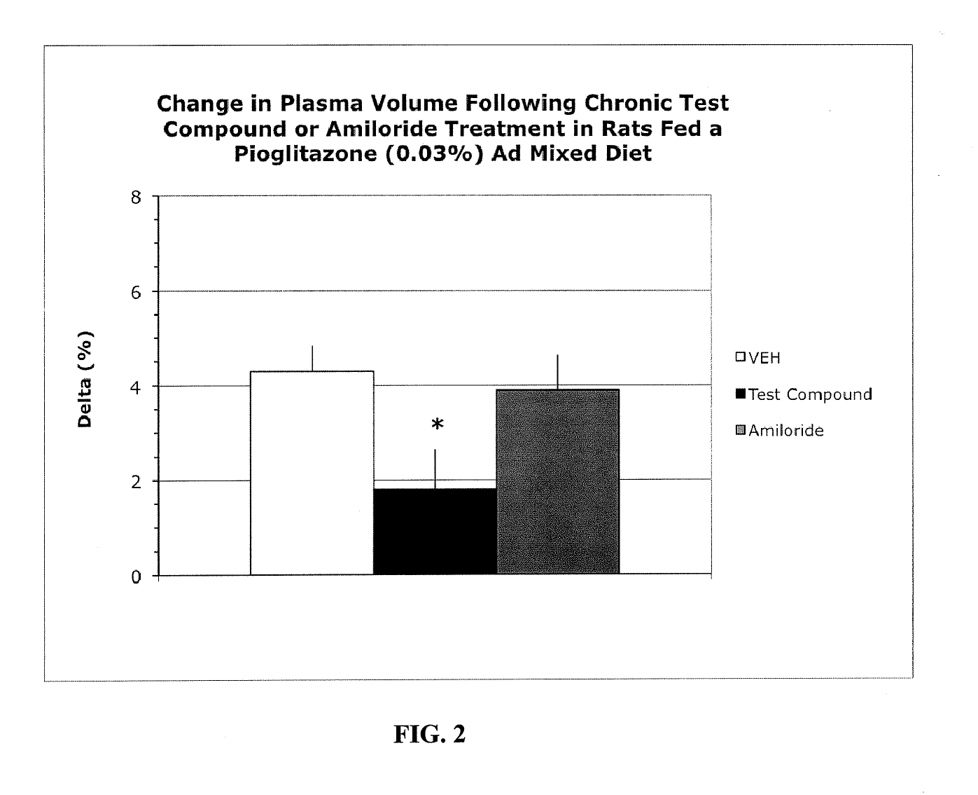

Significant effects of time (F_{(1,27)}=64.9; p<0.0001) and treatment (F_{(2,27)}=3.73; p=0.037), as well as a significant interaction (F_{(2,27)}=3.51; p=0.044) were revealed by the 2-way ANOVA. Day 1 mean plasma volumes were similar (approximately 53%) among the treatment groups (F_{(2,27)}=0.45; p=0.64). However, after consuming a pioglitazone containing diet for 7 days, plasma volumes were significantly different (F_{(2,27)}=7.32; p=0.003), with TEST COMPOUND treated rats displaying significantly less plasma expansion than vehicle treated rats (p≦0.01) (FIG. 1 and Table 1). Further, while day 7 plasma volumes were significantly increased compared to day 1 plasma volumes in rats receiving vehicle or amiloride (p<0.001), plasma expansion in TEST COMPOUND treated rats did not reach significance (FIG. 1 and Table 1). Analysis of the change in plasma volume expressed as a percentage of blood volume resulted in similar ANOVA results (F_{(2,27)}=3.51; p<0.044) (FIG. 2 and Table 1). Pair-wise comparisons of the change scores were also similar to those from the day 7 analyses, as TEST COMPOUND treated rats displayed significantly less plasma expansion than vehicle treated rats (p≦0.05).

### Body Weight:

Raw Weight Gain: A significant effect of time (F_{(1,27)}=293.4; p<0.0001) and a significant interaction (F_{(2,27)}=18.6; p<0.0001) were revealed by 2-way ANOVA. However, the Treatment effect failed to reach significance (F_{(2,27)}=2.11; p=0.14). As animals were assigned to treatment groups, counterbalanced against body weights, day 1 mean body weights (approximately 370 g) were similar (F_{(2,27)}=0.070; p=0.93). Weight gain was observed over the course of the experiment, with a significant difference among day 7 group means (F_{(2,27)}=6.05; p=0.007). All rats gained significant weight over the 7 days regardless of treatment (p<0.0001). However, those receiving TEST COMPOUND gained significantly less weight than those receiving vehicle; 17.4 g compared to 45.9 g (p≦0.01). Analysis of the weight gain from day 1 to day 7 resulted in similar ANOVA results (F_{(2,27)}=18.2; p<0.0001). Pair-wise comparisons were also similar to those from the day 7 analyses, as TEST COMPOUND treated rats displayed significantly less weight gain than vehicle treated rats (p≦0.01).

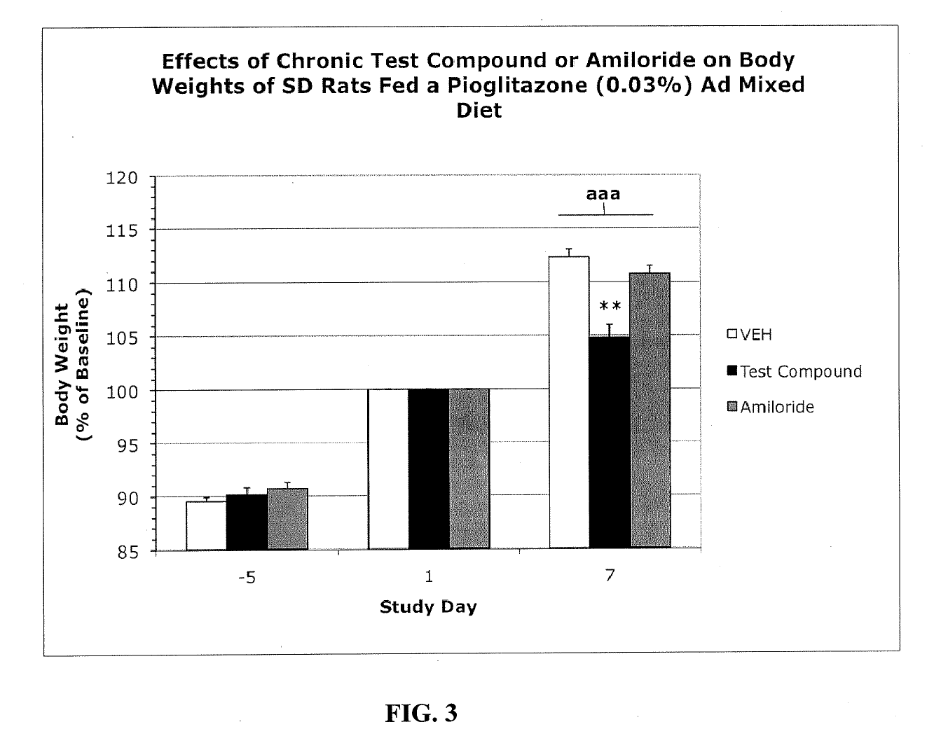

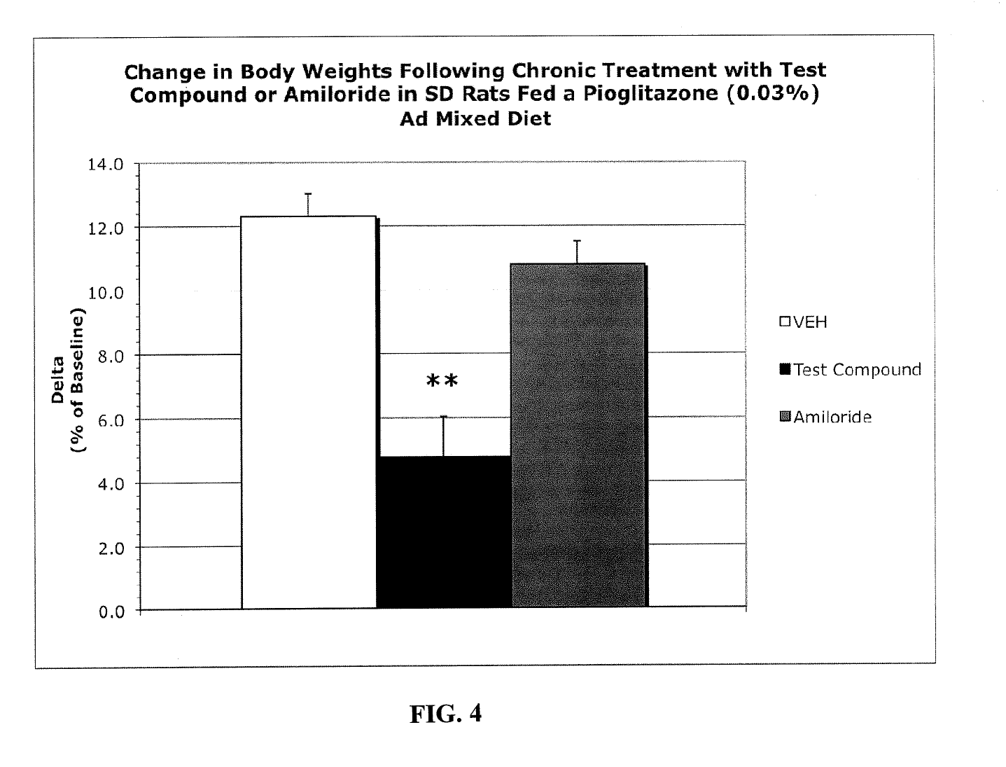

Percent Weight Gain: Significant effects of time (F_{(1,27)}=303.8; p<0.0001) and treatment (F_{(2,27)}=18.6; p<0.0001), as well as a significant interaction (F_{(2,27)}=18.6; p<0.0001) were revealed by 2-way ANOVA. Animals were assigned to treatment groups, counterbalanced against body weights, and weight gains were expressed as gains from day 1 values. Therefore, day 1 mean body weights were identical (100%). Weight gain was observed over the course of the experiment, with a significant difference among day 7 group means expressed as percent weight gain (F_{(2,27)}=18.6; p<0.0001) (FIG. 3 and Table 2). All rats gained significant weight over the 7 days irrespective of treatment (p<0.0001). However, those receiving TEST COMPOUND gained significantly less weight than those receiving vehicle; 4.8% gain compared to 12.3% gain (p≦0.01). Analysis of the differences from day 1 to day 7 resulted in similar ANOVA results (F_{(2,27)}=18.6; p<0.0001) (FIG. 4 and Table 2). Pair-wise comparisons were also similar to those from the day 7 analyses, as TEST COMPOUND treated rats displayed significantly less weight gain than vehicle treated rats (p≦0.01).

### Food Consumption/Pioglitazone Exposure:

Daily food consumption was similar, in all treatment groups, over the 7 days of the experiment. However, slightly lower consumption was observed in the amiloride treated rats and slightly higher consumption was observed in the TEST COMPOUND treated rats, resulting in a significant overall ANOVA (F_{(2,27)}=3.94; p=0.032). Neither treatment group was found to consume a significantly different amount of food than vehicle treated rats. As pioglitazone dosing was achieved by combining the compound in the feed, animals received similar levels of pioglitazone exposure.

### Water Consumption/Compound Exposure:

Daily water consumption was significantly different, across the treatment groups, over the 7 days of the experiment (F_{(2,27)}=53.8; p<0.0001). This was consistent with the known propensity for TEST COMPOUND to increase water consumption in rats; and the magnitude of the increase (>40%) compared to vehicle treated rats was significant (p≦0.01). Dosing concentrations were calculated with this effect in mind, although underestimated. Therefore, daily doses of TEST COMPOUND and amiloride received were slightly higher than the targeted range; approximately 4.5 mg/kg/day TEST COMPOUND and 1.5 mg/kg/day amiloride.

### Discussion

One week of treatment with the PPAR-γ agonist pioglitazone in the range of 20-25 mg/kg/day resulted in significant plasma expansion, as indicated by a reduction in the packed cell volume to plasma ratio. Additional evidence of fluid retention was provided by the significant increase in body weight (approximately 45 g) over this short interval, as it is well established that early PPAR-γ mediated weight gain is the result of fluid accumulation (Guan Y, et al., Nature Med 11:861-6, 2005). Both of these effects were ameliorated, although not completely prevented, by concurrent administration of the SGLT2 inhibitor TEST COMPOUND. The observed effects of TEST COMPOUND cannot be explained by either differences in caloric intake, or resultant dose of pioglitazone received, as food consumption was similar across treatment groups. Rats receiving TEST COMPOUND consumed more food, and hence received a higher dose of pioglitazone, than rats receiving vehicle, but these rats gained the least amount of weight and experienced the lowest amount of plasma expansion. Significantly increased water consumption was also observed in rats treated with TEST COMPOUND, in keeping with previously documented effects of this class of compounds. One effect of this increased consumption was that these rats received a slightly higher dose of TEST COMPOUND than was targeted (approximately 4.5 instead of 3.0 mg/kg/day). Although the sodium channel blocker amiloride was employed as a reference compound based on previous studies demonstrating blockade of volume expansion in mice (Guan Y, et al., Nature Med 11:861-6, 2005), significance for the observed trend toward reversal of PPAR-γ induced expansion was not achieved. The amiloride-exacerbated volume expansion observed in rats by Chen et al. (Chen L, et al., J Pharmacol Exp Ther 312:718-25, 2005) could not be confirmed.

### CONCLUSIONS

Concurrent oral treatment with the SGLT2 inhibitor TEST COMPOUND antagonized pioglitazone induced plasma expansion and body water retention, providing support for the hypothesis that these PPAR-γ mediated effects result from renal sodium retention.

The results of this study support further development of TEST COMPOUND as a candidate therapeutic to treat PPAR-γ-mediated fluid retention and weight gain in humans.

TABLE 1
Effects of TEST COMPOUND or Amiloride Treatment
on Plasma Volume of SD Rats Fed a Pioglitazone
(0.03%) Containing Diet for 7 Days

| Group | | Vehicle | TEST COMPOUND | Amiloride |
|---|---|---|---|---|
| Plasma | DAY 1 | 53.5 ± 0.4 | 52.9 ± 0.5 | 52.7 ± 0.9 |
| Volume | DAY 7 | 57.8 ± 0.7 ^{††} | 54.7 ± 0.6 ** | 56.6 ± 0.4 ^{††} |
| % | DELTA | 4.3 ± 0.5 | 1.8 ± 0.8 * | 3.9 ± 0.7 |

Values represent group means ± SEM;
* p ≦ 0.05,
** p ≦ 0.01 (vs Vehicle);
^{†} p ≦ 0.05,
^{††} p ≦ 0.01 (vs Day 1)
TEST COMPOUND (4.5 mg/kg) or amiloride (1.5 mg/kg) were administered through the drinking water. Values represent mean plasma volumes (%) ± 1 SEM. Day 1 represents values obtained on the day rats were first exposed to the pioglitazone containing diet and water bottles containing compounds. Differences (Deltas) were calculated by subtraction of day 1 values from day 7 values. Asterisks indicate significant differences from vehicle control (water) on the same day (* p < 0.05, ** p < 0.01) while crosses indicate significant differences from day 1 (^{†} p < 0.05, ^{††} p < 0.01).

TABLE 2
Effects of TEST COMPOUND or Amiloride Treatment
on Body Weights of SD Rats Fed a Pioglitazone
(0.03%) Containing Diet for 7 Days

| Group | | Vehicle | TEST COMPOUND | Amiloride |
|---|---|---|---|---|
| Body | DAY 1 | 372.8 ± 3.5 | 371.7 ± 5.0 | 370.0 ± 6.0 |
| Weight | DAY 7 | 418.7 ± 4.6 ^{††} | 389.2 ± 5.7 ** ^{††} | 410.0 ± 7.7 ^{††} |
| (g) | DELTA | 45.9 ± 2.6 | 17.5 ± 4.7 ** | 40.0 ± 2.8 |
| Body | DAY 1 | 100 ± 0 | 100 ± 0 | 100 ± 0 |
| Weight | DAY 7 | 112.3 ± 0.7 ^{††} | 104.8 ± 1.3 ** ^{††} | 110.8 ± 0.7 ^{††} |
| (%) | DELTA | 12.3 ± 0.7 | 4.8 ± 1.3 ** | 10.8 ± 0.7 |

Values represent group means ± SEM;
* p ≦ 0.05,
** p ≦ 0.01 (vs Vehicle);
^{†} p ≦ 0.05,
^{††} p ≦ 0.01 (vs Day 1)
TEST COMPOUND (4.5 mg/kg) or amiloride (1.5 mg/kg) were administered through the drinking water. Values represent means ± 1 SEM. Day 1 represents values obtained on the day rats were first exposed to the pioglitazone containing diet and water bottles containing compounds. Differences (Deltas) were calculated by subtraction of day 1 values from day 7 values. Asterisks indicate significant differences from vehicle control (water) on the same day (* p < 0.05, ** p < 0.01) while crosses indicate significant differences from day 1 (^{†} p < 0.05, ^{††} p < 0.01).

What is claimed is:

1. A method for the reduction of fluid retention, comprising administering to a subject in need thereof a therapeutically effective amount of a sodium-dependent glucose transporter 2 inhibitor (SGLT2 inhibitor).

2. The method of claim 1, wherein said fluid retention is associated with administration of a PPAR-gamma agonist to said subject.

3. The method of claim 2, wherein said PPAR-gamma agonist is a member selected from the group consisting of rosiglitazone, pioglitazone, rivoglitazone, netoglitazone and THR-0921.

4. The method of claim 2, wherein said subject is at risk for development of a disease or condition associated with fluid retention.

5. The method of claim 2, further comprising administering an effective amount of a diuretic.

6. The method of claim 5, wherein said diuretic is a member selected from the group consisting of loop diuretics and thiazide and thiazide-like diuretics.

7. A method for the treatment of type 2 diabetes, comprising administering to a subject in need thereof, a therapeutically effective amount of a combination of an SGLT2 inhibitor and a PPAR-gamma agonist, wherein said subject is at risk for development of a disease or condition associated with fluid retention.

8. The method of claim 7, further comprising administering an effective amount of a diuretic.

9. The method of claim 8, wherein said diuretic is a member selected from the group consisting of loop diuretics and thiazide and thiazide-like diuretics.

10. A method for the treatment of type 2 diabetes, comprising administering to a subject in need thereof, a therapeutically effective amount of a combination of an SGLT2 inhibitor and a PPAR-gamma agonist, wherein said subject has a disease or condition associated with fluid retention.

11. The method of claim 10, wherein said PPAR-gamma agonist is a member selected from the group consisting of rosiglitazone, pioglitazone, rivoglitazone, netoglitazone and THR-0921.

12. The method of claim 10, further comprising administering an effective amount of a diuretic.

13. The method of claim 12, wherein said diuretic is a member selected from the group consisting of loop diuretics and thiazide and thiazide-like diuretics.

14. The method of claim 7, wherein said SGLT2 inhibitor is a compound of Formula I: 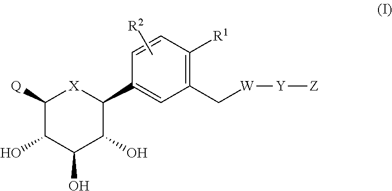
or a pharmaceutically acceptable salt thereof, wherein,
X is a member selected from the group consisting of oxygen and sulfur;
Q is a member selected from the group consisting of —CH₂OH, C₁-C₆ alkylsulfanyl, C₁-C₆ alkylsulfinyl, C₁-C₆ alkylsulfonyl, C₁-C₆ haloalkylsulfanyl, C₁-C₆ haloalkylsulfinyl, C₁-C₆ haloalkylsulfonyl, and —CH₂OV, wherein V is a member selected from the group consisting of (C₁-C₃ alkyl)oxycarbonyl, (C₁-C₆ alkyl)carbonyl, phenyloxycarbonyl, benzylcarbonyl and benzyloxycarbonyl;
R¹ and R² are each members independently selected from the group consisting of hydrogen, halo, C₁-C₃ alkyl, C₂-C₃ alkynyl, C₃-C₆ cycloalkyl, hydroxy and cyano;
W is a member selected from the group consisting of a 5- to 6-membered aryl or heteroaryl ring, and an 8- to 10-membered fused bicyclic aryl or heteroaryl ring, wherein W optionally may be mono- or disubstituted by identical or different substituents selected from the group consisting of halo, hydroxy, C₁-C₃ alkyl, C₁-C₃ alkoxy, cyano, —NR^{a}R^{b}, —C(O)NR^{a}R^{b}, C₁-C₆ alkylsulfanyl, C₁-C₆ alkylsulfinyl, and C₁-C₆ alkylsulfonyl, and wherein alkyl groups or portions are optionally partly or completely fluorinated;
Y is a member selected from the group consisting of a single bond and a 5- to 6-membered aryl or heteroaryl ring, wherein Y is optionally mono- or disubstituted by identical or different substituents selected from the group consisting of halo, hydroxy, C₁-C₃ alkyl, C₁-C₃ alkoxy, cyano, —NR^{a}R^{b}, —C(O)NR^{a}R^{b}, C₁-C₆ alkylsulfanyl, C₁-C₆ alkylsulfinyl and C₁-C₆ alkylsulfonyl, and wherein alkyl groups or portions are optionally partly or completely fluorinated;
Z is a member selected from the group consisting of hydrogen, halo, hydroxy, cyano, C₁-C₃ alkyl, C₁-C₃ alkoxy, C₂-C₃ alkynyl, C₃-C₆ cycloalkyl, C₃-C₆ heterocycloalkyl, C₃-C₆ cycloalkoxy, C₃-C₆ heterocycloalkoxy, (C₁-C₃ alkoxy)C₁-C₃ alkoxy and (C₃-C₆ cycloalkoxy)C₁-C₃ alkoxy, wherein alkyl, alkynyl, cycloalkyl and heterocycloalkyl groups or portions are optionally partly or completely fluorinated and are optionally mono- or disubstituted by identical or different substituents selected from the group consisting of chloro, hydroxy, C₁-C₃ alkyl, C₁-C₃ alkoxy, cyano, —NR^{a}R^{b}, —C(O)NR^{a}R^{b}, C₁-C₆ alkylsulfanyl, C₁-C₆ alkylsulfinyl and C₁-C₆ alkylsulfonyl;
R^{a} and R^{b} are each members independently selected from the group consisting of hydrogen and C₁-C₆ alkyl, wherein the alkyl groups are optionally partly or completely fluorinated; and
wherein optionally one or more hydrogen atoms may be substituted with deuterium.

15. The method of claim 14, wherein Q is a member selected from the group consisting of —CH₂OH and methylsulfonyl; R¹ is a member selected from the group consisting of hydrogen, chloro, fluoro, methyl and cyano; R² is a member selected from the group consisting of hydrogen and hydroxy; W is phenyl; Y is a single bond; and Z is a member selected from the group consisting of ethyl, ethoxy, ethynyl, cyclopropyl, benzo[b]thiophen-2-yl, azulenyl, tetrahydrofuran-3-yloxy and cyclopropoxyethoxy.

16. The method of claim 14, wherein said compound is selected from the group consisting of:
(2S,3R,4R,5S,6R)-2-(4-chloro-3-(4-ethoxybenzyl)phenyl)-6-(hydroxymethyl)tetrahydro-2H-pyran-3,4,5-triol;
(2S,3R,4R,5S,6R)-2-(4-chloro-3-(4-ethylbenzyl)phenyl)-6-(hydroxymethyl)tetrahydro-2H-pyran-3,4,5-triol;
(2S,3R,4R,5S,6R)-2-(4-chloro-3-(4-ethynylbenzyl)phenyl)-6-(hydroxymethyl)tetrahydro-2H-pyran-3,4,5-triol;
(2S,3R,4R,5S,6R)-2-(4-chloro-3-(4-cyclopropylbenzyl)phenyl)-6-(hydroxymethyl)tetrahydro-2H-pyran-3,4,5-triol;
2S,3R,4R,5S,6R)-2-(4-chloro-3-(4-(ethoxy-d₅)benzyl)phenyl)-6-(hydroxymethyl)tetrahydro-2H-pyran-3,4,5-triol;
(2S,3R,4R,5S,6R)-2-(4-chloro-3-((4-cyclopropylphenyl)methyl-d₂)phenyl)-6-(hydroxymethyl)tetrahydro-2H-pyran-3,4,5-triol;
(2S,3R,4R,5S,6R)-2-(4-chloro-3-(4-((S)-tetrahydrofuran-3-yloxy)benzyl)phenyl)-6-(hydroxymethyl)tetrahydro-2H-pyran-3,4,5-triol;
(2S,3R,4R,5S,6R)-2-(4-chloro-3-(4-((R)-tetrahydrofuran-3-yloxy)benzyl)phenyl)-6-(hydroxymethyl)tetrahydro-2H-pyran-3,4,5-triol;
(2S,3R,4R,5S,6R)-2-(4-chloro-3-(4-(2-cyclopropoxyethoxy)benzyl)phenyl)-6-(hydroxymethyl)tetrahydro-2H-pyran-3,4,5-triol;
(2S,3R,4R,5S,6R)-2-(4-chloro-3-(4-ethoxybenzyl)phenyl)-6-(methylsulfonyl)tetrahydro-2H-pyran-3,4,5-triol;
(2S,3R,4R,5S,6R)-2-(4-chloro-3-(4-ethoxybenzyl)phenyl)-6-(methylthio)tetrahydro-2H-pyran-3,4,5-triol;
(2S,3R,4R,5S,6R)-2-(3-(benzo[b]thiophen-2-ylmethyl)-4-fluorophenyl)-6-(hydroxymethyl)tetrahydro-2H-pyran-3,4,5-triol;
(2S,3R,4R,5S,6R)-2-(5-(azulen-2-ylmethyl)-2-hydroxyphenyl)-6-(hydroxymethyl)tetrahydro-2H-pyran-3,4,5-triol;
(2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol;
(2S,3R,4R,5S,6R)-2-(4-chloro-3-(4-ethoxybenzyl)phenyl)-6-(hydroxymethyl)tetrahydro-2H-thiopyran-3,4,5-triol;
(2S,3R,4R,5S,6R)-2-(3-(benzo[b]thiophen-2-ylmethyl)-4-chlorophenyl)-6-(hydroxymethyl)tetrahydro-2H-thiopyran-3,4,5-triol; and
(2S,3R,4R,5S,6R)-2-(5-(4-ethoxybenzyl)-2-methoxy-4-methylphenyl)-6-(hydroxymethyl)tetrahydro-2H-thiopyran-3,4,5-triol.

17. The method of claim 10, wherein said SGLT2 inhibitor is a compound of Formula I: 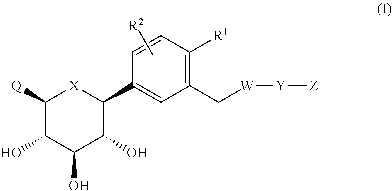
or a pharmaceutically acceptable salt thereof, wherein,
X is a member selected from the group consisting of oxygen and sulfur;
Q is a member selected from the group consisting of —CH₂OH, C₁-C₆ alkylsulfanyl, C₁-C₆ alkylsulfinyl, C₁-C₆ alkylsulfonyl, C₁-C₆ haloalkylsulfanyl, C₁-C₆ haloalkylsulfinyl, C₁-C₆ haloalkylsulfonyl, and —CH₂OV, wherein V is a member selected from the group consisting of (C₁-C₃ alkyl)oxycarbonyl, (C₁-C₆ alkyl)carbonyl, phenyloxycarbonyl, benzylcarbonyl and benzyloxycarbonyl;
R¹ and R² are each members independently selected from the group consisting of hydrogen, halo, C₁-C₃ alkyl, C₂-C₃ alkynyl, C₃-C₆ cycloalkyl, hydroxy and cyano;
W is a member selected from the group consisting of a 5- to 6-membered aryl or heteroaryl ring, and an 8- to 10-membered fused bicyclic aryl or heteroaryl ring, wherein W optionally may be mono- or disubstituted by identical or different substituents selected from the group consisting of halo, hydroxy, C₁-C₃ alkyl, C₁-C₃ alkoxy, cyano, —NR^{a}R^{b}, —C(O)NR^{a}R^{b}, C₁-C₆ alkylsulfanyl, C₁-C₆ alkylsulfinyl, and C₁-C₆ alkylsulfonyl, and wherein alkyl groups or portions are optionally partly or completely fluorinated;
Y is a member selected from the group consisting of a single bond and a 5- to 6-membered aryl or heteroaryl ring, wherein Y is optionally mono- or disubstituted by identical or different substituents selected from the group consisting of halo, hydroxy, C₁-C₃ alkyl, C₁-C₃ alkoxy, cyano, —NR^{a}R^{b}, —C(O)NR^{a}R^{b}, C₁-C₆ alkylsulfanyl, C₁-C₆ alkylsulfinyl and C₁-C₆ alkylsulfonyl, and wherein alkyl groups or portions are optionally partly or completely fluorinated;
Z is a member selected from the group consisting of hydrogen, halo, hydroxy, cyano, C₁-C₃ alkyl, C₁-C₃ alkoxy, C₂-C₃ alkynyl, C₃-C₆ cycloalkyl, C₃-C₆ heterocycloalkyl, C₃-C₆ cycloalkoxy, C₃-C₆ heterocycloalkoxy, (C₁-C₃ alkoxy)C₁-C₃ alkoxy and (C₃-C₆ cycloalkoxy)C₁-C₃ alkoxy, wherein alkyl, alkynyl, cycloalkyl and heterocycloalkyl groups or portions are optionally partly or completely fluorinated and are optionally mono- or disubstituted by identical or different substituents selected from the group consisting of chloro, hydroxy, C₁-C₃ alkyl, C₁-C₃ alkoxy, cyano, —NR^{a}R^{b}, —C(O)NR^{a}R^{b}, C₁-C₆ alkylsulfanyl, C₁-C₆ alkylsulfinyl and C₁-C₆ alkylsulfonyl;
R^{a} and R^{b} are each members independently selected from the group consisting of hydrogen and C₁-C₆ alkyl, wherein the alkyl groups are optionally partly or completely fluorinated; and
wherein optionally one or more hydrogen atoms may be substituted with deuterium.

18. The method of claim 17, wherein Q is a member selected from the group consisting of —CH₂OH and methylsulfonyl; R¹ is a member selected from the group consisting of hydrogen, chloro, fluoro, methyl and cyano; R² is a member selected from the group consisting of hydrogen and hydroxy; W is phenyl; Y is a single bond; and Z is a member selected from the group consisting of ethyl, ethoxy, ethynyl, cyclopropyl, benzo[b]thiophen-2-yl, azulenyl, tetrahydrofuran-3-yloxy and cyclopropoxyethoxy.

19. The method of claim 17, wherein said compound is selected from the group consisting of:
(2S,3R,4R,5S,6R)-2-(4-chloro-3-(4-ethoxybenzyl)phenyl)-6-(hydroxymethyl)tetrahydro-2H-pyran-3,4,5-triol;
(2S,3R,4R,5S,6R)-2-(4-chloro-3-(4-ethylbenzyl)phenyl)-6-(hydroxymethyl)tetrahydro-2H-pyran-3,4,5-triol;
(2S,3R,4R,5S,6R)-2-(4-chloro-3-(4-ethynylbenzyl)phenyl)-6-(hydroxymethyl)tetrahydro-2H-pyran-3,4,5-triol;
(2S,3R,4R,5S,6R)-2-(4-chloro-3-(4-cyclopropylbenzyl)phenyl)-6-(hydroxymethyl)tetrahydro-2H-pyran-3,4,5-triol;
2S,3R,4R,5S,6R)-2-(4-chloro-3-(4-(ethoxy-d₅)benzyl)phenyl)-6-(hydroxymethyl)tetrahydro-2H-pyran-3,4,5-triol;
(2S,3R,4R,5S,6R)-2-(4-chloro-3-((4-cyclopropylphenyl)methyl-d₂)phenyl)-6-(hydroxymethyl)tetrahydro-2H-pyran-3,4,5-triol;
(2S,3R,4R,5S,6R)-2-(4-chloro-3-(4-((S)-tetrahydrofuran-3-yloxy)benzyl)phenyl)-6-(hydroxymethyl)tetrahydro-2H-pyran-3,4,5-triol;
(2S,3R,4R,5S,6R)-2-(4-chloro-3-(4-((R)-tetrahydrofuran-3-yloxy)benzyl)phenyl)-6-(hydroxymethyl)tetrahydro-2H-pyran-3,4,5-triol;
(2S,3R,4R,5S,6R)-2-(4-chloro-3-(4-(2-cyclopropoxyethoxy)benzyl)phenyl)-6-(hydroxymethyl)tetrahydro-2H-pyran-3,4,5-triol;
(2S,3R,4R,5S,6R)-2-(4-chloro-3-(4-ethoxybenzyl)phenyl)-6-(methylsulfonyl)tetrahydro-2H-pyran-3,4,5-triol;
(2S,3R,4R,5S,6R)-2-(4-chloro-3-(4-ethoxybenzyl)phenyl)-6-(methylthio)tetrahydro-2H-pyran-3,4,5-triol;
(2S,3R,4R,5S,6R)-2-(3-(benzo[b]thiophen-2-ylmethyl)-4-fluorophenyl)-6-(hydroxymethyl)tetrahydro-2H-pyran-3,4,5-triol;
(2S,3R,4R,5S,6R)-2-(5-(azulen-2-ylmethyl)-2-hydroxyphenyl)-6-(hydroxymethyl)tetrahydro-2H-pyran-3,4,5-triol;
(2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol;
(2S,3R,4R,5S,6R)-2-(4-chloro-3-(4-ethoxybenzyl)phenyl)-6-(hydroxymethyl)tetrahydro-2H-thiopyran-3,4,5-triol;
(2S,3R,4R,5S,6R)-2-(3-(benzo[b]thiophen-2-ylmethyl)-4-chlorophenyl)-6-(hydroxymethyl)tetrahydro-2H-thiopyran-3,4,5-triol; and
(2S,3R,4R,5S,6R)-2-(5-(4-ethoxybenzyl)-2-methoxy-4-methylphenyl)-6-(hydroxymethyl)tetrahydro-2H-thiopyran-3,4,5-triol.
